(19) United States
(12) Patent Application Publication
(10) Pub. No.: US 2026/0311827 A1
(43) Pub. Date: Oct. 8, 2026

(54) SYSTEMS AND METHODS FOR RADIATION THERAPY MANAGEMENT

(71) Applicant: GE Precision Healthcare LLC, Waukesha, WI (US)

(72) Inventors: Tripti Gupta, Sussex, WI (US); Sampath Telikicherla Kandala, Marlborough, MA (US); Timothy Lin, Brookfield, WI (US)

(21) Appl. No.: 19/483,155

(22) PCT Filed: May 10, 2024
(86) PCT No.: PCT/US2024/028969

§ 371 Date: Nov. 10, 2025

Related U.S. Application Data
(60) Provisional application No. 63/501,581, filed on May 11, 2023.

(51) Int. Cl. G16H 10/60; G16H 20/40

(52) U.S. Cl. CPC G16H 10/60; G16H 20/40

(57) ABSTRACT

Various methods and systems are provided for radiation therapy management. In one example, a computing device comprises a display screen, the computing device being configured to display on the display screen a course directive graphical user interface (GUI). The course directive GUI displays, for a selected patient, a limited list of patient information obtained from one or more of an electronic medical record (EMR) database and an oncology information system (OIS), and further displays a limited list of radiation therapy forms each being selectable to launch a display panel and enable at least the selected form to be seen within the display panel.

### CROSS REFERENCE TO RELATED APPLICATIONS

This application claims priority to U.S. Provisional Application No. 63/501,581, titled “SYSTEMS AND METHODS FOR RADIATION THERAPY MANAGEMENT”, and filed May 11, 2023, the entire contents of which is hereby incorporated by reference for all purposes.

### FIELD

Embodiments of the subject matter disclosed herein relate to radiation therapy, and more particularly to a graphical user interface for displaying integrated radiation therapy data.

### BACKGROUND

Radiation oncology may include the administration of targeted radiation to a patient's tumor, for example. To reduce radiation incident on nearby healthy tissue, precise radiation planning may be performed where the patient is first scanned with a medical imaging device (e.g., computed tomography scanner) to build a 3D model of the patient's tumor. The radiation therapy may be delivered to the tumor based on the 3D model, in one or more phases and at one or more targets. Thus, multiple care providers may be involved in the planning and administration of the radiation therapy, with information stored and retrieved from various siloed data sources.

### BRIEF DESCRIPTION

In one example, a computing device comprises a display screen, the computing device being configured to display on the display screen a course directive graphical user interface (GUI) for radiation therapy (RT) management, wherein the course directive GUI displays, for a selected patient, a limited list of patient information, the limited list of patient information obtained from one or more of an electronic medical record (EMR) database and an oncology information system (OIS), the course directive GUI further displaying a limited list of radiation therapy forms, each radiation therapy form in the limited list of radiation therapy forms being selectable to launch a display panel and enable at least the selected form to be seen within the display panel.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows a block diagram of an example computing system.

FIG. 2 shows a block diagram of an example integrated radiation oncology system.

FIG. 3 shows an example course directive graphical user interface (GUI) in a first view.

FIG. 4 shows the course directive GUI in a second view.

FIG. 5 shows the course directive GUI in the second view with a display panel including a simulation order form launched.

FIG. 6 shows the course directive GUI in a third view.

FIG. 7 shows the course directive GUI in a fourth view.

FIG. 8 shows an example multi-patient GUI in a first view.

FIG. 9 shows the multi-patient GUI in a second view.

FIG. 10 shows another example course directive GUI.

FIG. 11 shows an example template management GUI in a first view.

FIG. 12 shows the template management GUI in a second view.

FIG. 13 is a flowchart illustrating a method for integrating radiation therapy data and displaying the integrated data via a course directive GUI.

FIG. 14 is a flowchart illustrating a method for managing a radiation therapy workflow via a multi-patient GUI.

FIG. 15 is a flowchart illustrating a method for managing templates via a template management GUI.

FIG. 16 is a block diagram showing an example automated image workflow according to embodiments of the disclosure.

### DETAILED DESCRIPTION

Radiation oncology includes the administration of radiation therapy to a target volume, such as a patient's tumor. The radiation therapy may be delivered via a radiation therapy device, such as a linear accelerator. In order to precisely radiate the target while sparing surrounding tissue, radiation therapy may be guided with images and/or 3D models of the target obtained with a medical imaging device, such as a computed tomography (CT) scanner, magnetic resonance imaging (MRI) scanner, positron emission tomography (PET) scanner, or ultrasound device. Thus, the process of planning radiation therapy for a given patient may include planning the medical imaging exam and planning the delivery of the radiation therapy.

The planning of the two separate but related procedures (e.g., medical imaging and radiation therapy delivery) carried out with separate devices may dictate that a clinician plan the medical imaging exam (also referred to as a simulation scan when performed in the context of radiation therapy planning) by manually entering patient information and imaging exam parameters into a first interface (e.g., of the medical imaging device). Then, the clinician (or another clinician) may plan the radiation therapy session(s) by manually entering patient information and radiation therapy parameters into a second interface (e.g., of the radiation therapy device). Further, after the imaging exam is performed, a user may manually search for the imaging exam in an image archive, select images from the imaging exam, and direct the subsequent use of the selected images, such as sending the selected images to a particular device for segmentation and/or contouring. Similarly, once the images have been segmented and/or contoured, the same user or another user may manually search for the segmented and/or contoured images for review and direct the subsequent use of the segmented and/or contoured images, such as sending the segmented and/or contoured images to a radiation therapy device. This process may be time-consuming and demand performance of redundant actions, such as entering the same patient information into multiple planning forms. Relying on manual look-up and entry of information and manual look-up and directing of images may also induce errors in the treatment planning and unduly tie up resources.

Thus, according to embodiments disclosed herein, a radiation therapy management system may integrate data from multiple different sources, auto-populate planning forms with known information, and present radiation therapy planning forms on a single interface, referred to as a course directive graphical user interface (GUI) for radiation therapy (RT) management, which may be reached from a multi-patient workflow management GUI (also referred to as a multi-patient GUI). The course directive GUI may provide a limited list of information relevant to radiation therapy planning, including a limited list of relevant patient information and a limited list of radiation therapy planning forms. The patient information may be auto-filled by the radiation therapy management system based on information already stored in separate, siloed data sources, such as an electronic medical record (EMR) database and an oncology information systems (OIS). Likewise, the radiation therapy planning forms (which may include a simulation scan planning form and a radiation therapy planning form) may be auto-populated with information from the EMR database and OIS, as well as auto-populated with information obtained from user input during the planning process. For example, a clinician, when planning the simulation scan, may select a diagnosis, scanning protocol, etc., and this information (where relevant) may be used to populate the simulation scan planning form and/or radiation therapy planning form. Further, the radiation therapy planning forms may be formatted and auto-filled according to a selected template when desired, and templates may be created and edited via a template management GUI. Further, the radiation therapy management system may send the completed simulation scan planning form to a medical imaging device for carrying out the simulation scan and may send the completed radiation therapy planning form to a radiation therapy device for carrying out the radiation therapy. The overall workflow for radiation therapy planning and delivery for a plurality of patients may be tracked via the multi-patient GUI, which may display prompts for users to perform activities of the workflow and allow the users to perform at least some of the activities directly via the multi-patient GUI. For example, via the multi-patient GUI, a user may view images obtained during the simulation scan and select images from the simulation scan for a downstream task, such as further review by a different user and/or to be sent to a contouring module and/or segmentation module. The RT management system may be configured to automatically perform a query to an image archive in order to retrieve the images obtained during the simulation scan and automatically send the selected images to a selected device for performing the downstream task, obviating the need for the user to have to manually search for the images on the image archive and manually indicate where the selected images are to be sent.

In this way, the two-stage process of radiation therapy planning may be integrated into a single course directive interface with known information populated by the radiation therapy management system, which may reduce user errors and increase a speed at which the radiation therapy planning may be conducted. Further, the activities to be carried out over the course of radiation therapy planning and delivery may be tracked and performed via a single multi-patient interface, which may reduce errors in image selection and transmission and increase the speed at which the radiation therapy planning may be conducted. The performance of the individual computing devices associated with the medical imaging device, image archive, and radiation therapy device may be improved by reducing the processing demands on those devices, owing to reduced user errors (and hence reduced scan retakes, for example) and reduced entry of redundant information.

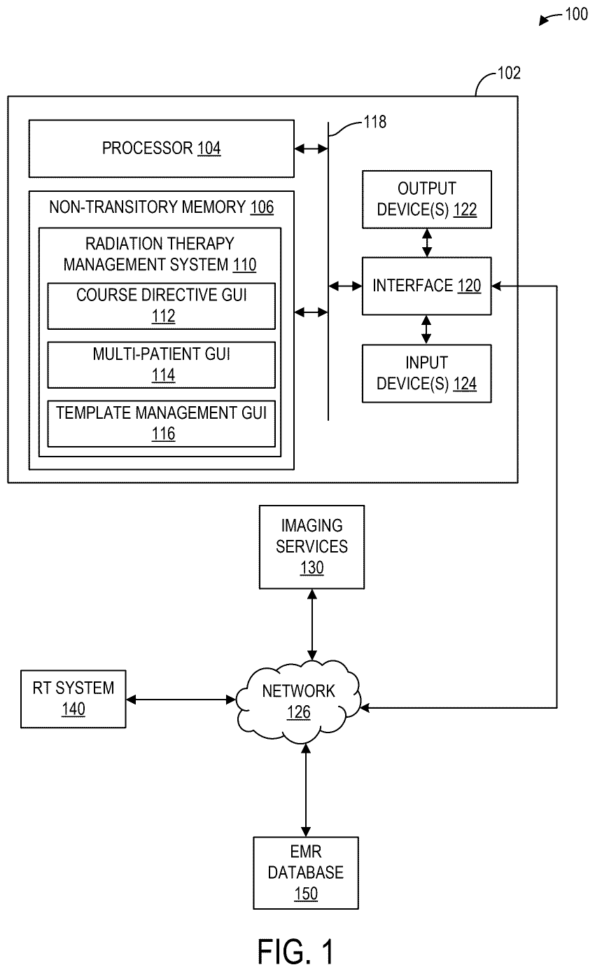

Referring now to FIG. 1, an example of a computing system 100 is shown. Computing system 100 comprises a computing device 102 which may comprise, as illustrative and non-limiting examples, a server, a personal computer, a workstation, a mobile device (e.g., a cellular phone, a smart phone, a computing tablet, and so on), or any other type of computing device.

The computing device 102 includes one or more processors 104 which may be configured to execute machine-readable instructions stored in non-transitory memory 106. Processor(s) 104 may be single core or multi-core, and the programs executed thereon may be configured for parallel or distributed processing. In some embodiments, the processor(s) 104 may optionally include individual hardware components that are distributed throughout two or more devices, which may be remotely located and/or configured for coordinated processing. In some embodiments, one or more aspects of the processor(s) 104 may be virtualized and executed by remotely-accessible networked computing devices configured in a cloud computing configuration. The computing device 102 further includes non-transitory memory 106. It should be appreciated that the computing device 102 may include additional memory devices, including volatile memory, mass storage, local memory, and so on.

Memory 106 includes one or more data storage structures, such as optical memory devices, magnetic memory devices, or solid-state memory devices, for storing programs and routines executed by processor(s) 104 to carry out various functionalities disclosed herein. Memory 106 may include any desired type of volatile and/or non-volatile memory such as, for example, static random access memory (SRAM), dynamic random access memory (DRAM), flash memory, read-only memory (ROM), etc. Processor(s) 104 may be any suitable processor, processing unit, or microprocessor, for example. Processor(s) 104 may be a multi-processor system, and, thus, may include one or more additional processors that are identical or similar to each other and that are communicatively coupled via an interconnection bus. In some examples, memory 106 may include components disposed at two or more devices, which may be remotely located and/or configured for coordinated processing. In some embodiments, one or more aspects of memory 106 may include remotely-accessible networked storage devices configured in a cloud computing configuration. The processor(s) 104 and memory 106 may be coupled, for example, via a communications bus 118.

The computing device 102 may further include an interface 120 communicatively coupled to the processor(s) 104 and memory 106 via the communications bus 118. The interface 120 may be implemented by one or more of any type of interface standard, such as an Ethernet interface, a universal serial bus (USB), a BLUETOOTH interface, a near field communication (NFC) interface, and/or a PCI express interface.

The computing device 102 may further include one or more output device(s) 122 communicatively coupled to the processor 104 and the non-transitory memory 106 via the interface 120. The output device(s) 122 may comprise, for example, one or more display devices. Such a display device may include one or more display devices utilizing virtually any type of technology (e.g., a light emitting diode (LED), an organic light emitting diode (OLED), a liquid crystal display (LCD), a cathode ray tube (CRT) display, an in-place switching (IPS) display, a touchscreen, and so on). In some examples, output device 122 may comprise a computer monitor configured to display medical information of various types and styles. Output device(s) 122 may be combined with processor(s) 104, memory 106, and/or user input device(s) 124 in a shared enclosure, or may be a peripheral display device and may comprise a monitor, touchscreen, projector, or other output device known in the art, which may enable a user to plan and administer radiation therapy according to one or more examples of the current disclosure, and/or interact with various data stored in memory 106.

The computing device 102 may further include one or more user input device(s) 124 coupled to the processor(s) 104 and memory 106 via the interface 120. A user input device 124 may comprise, for example, one or more of a touchscreen, a keyboard, a mouse, a trackpad, a motion sensing camera, a microphone, or other device configured to enable a user to interact with and manipulate data within computing device 102.

The interface 120 may further include a communication device such as a transmitter, a receiver, a transceiver, a modem, a residential gateway, a wireless access point, and/or a network interface to facilitate exchange of data with external machines (e.g., computing devices of any kind) via a network 126. For example, the communication may be via, for example, an Ethernet connection, a digital subscriber line (DSL) connection, a telephone line connection, a coaxial cable system, a satellite system, a line-of-site wireless system, a cellular telephone system, and so on. As a non-limiting example, FIG. 1 shows a plurality of devices/systems that may be communicatively coupled to computing device 102 via network 126, including imaging services 130, a radiation therapy (RT) system 140, and an EMR database 150.

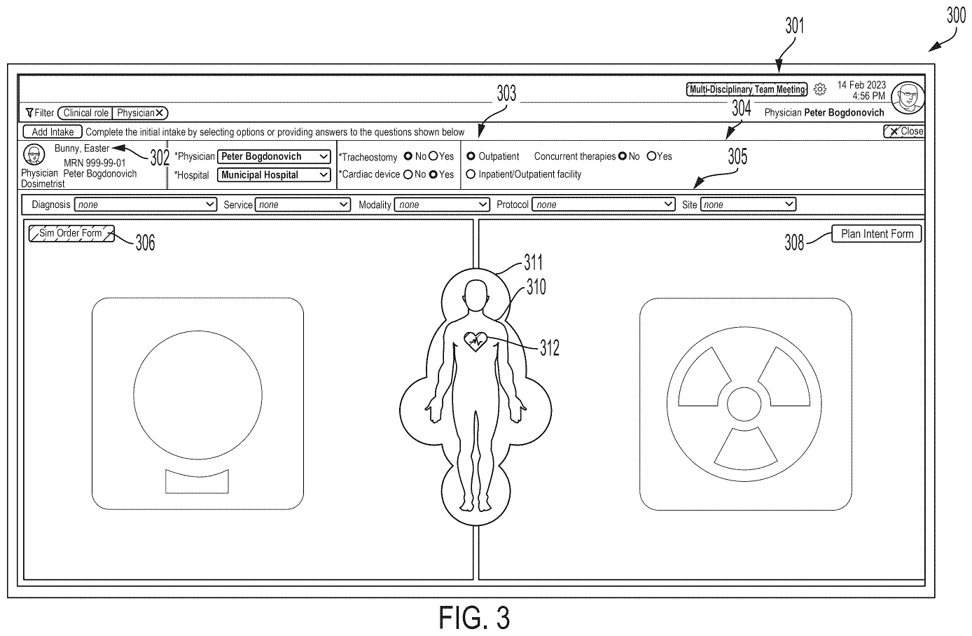

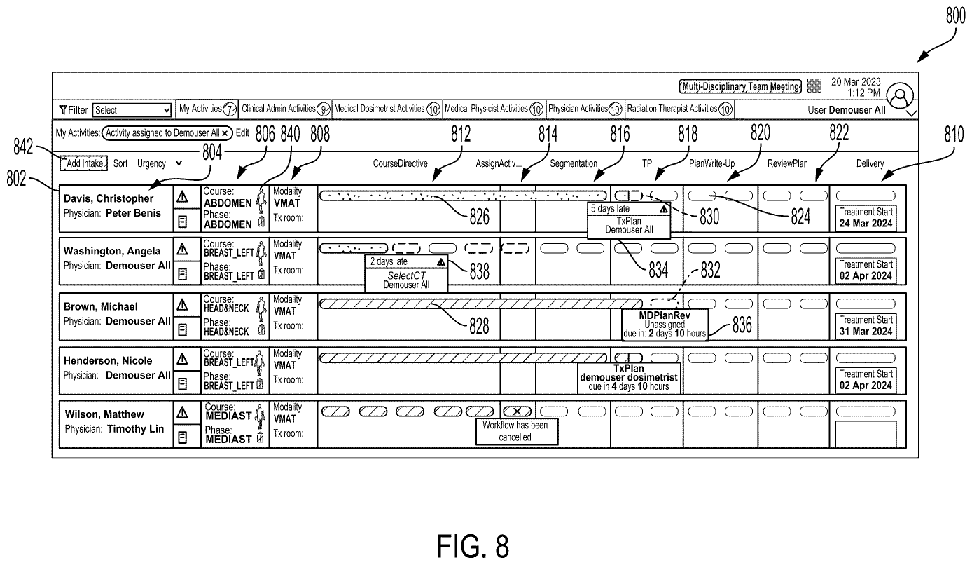

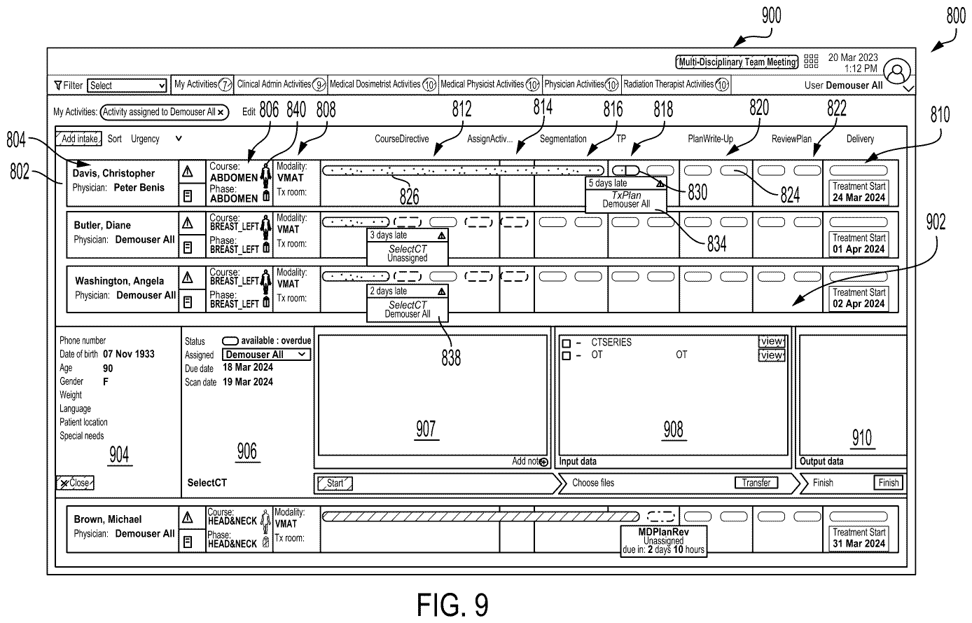

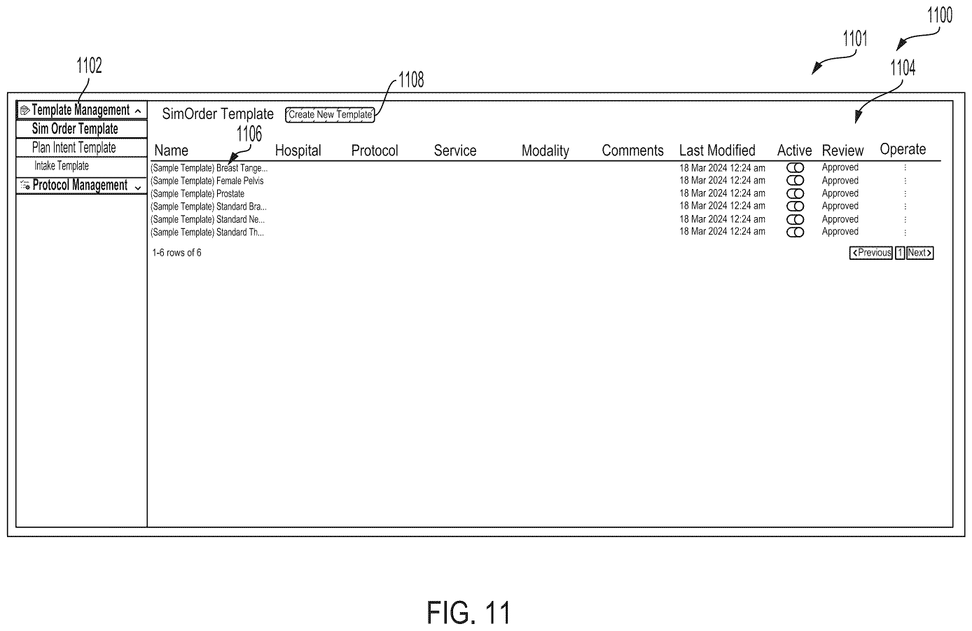

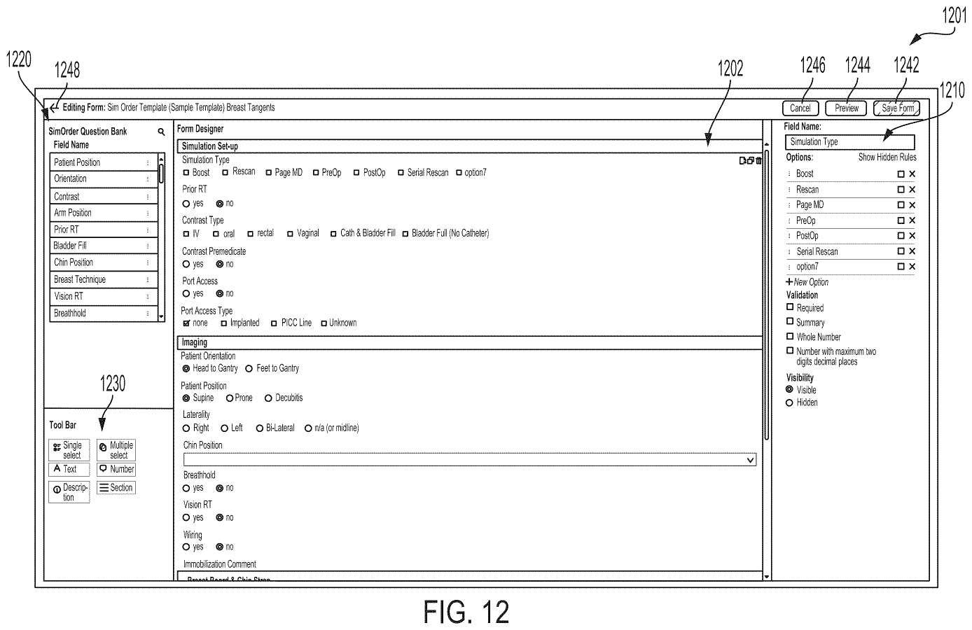

Computing device 102 may further include a radiation therapy management system 110 saved in memory 106. The radiation therapy management system 110 may be configured to obtain information from various data sources and display the information as part of plurality of graphical user interfaces (GUIs) for radiation therapy (RT) management, including a course directive GUI 112, a multi-patient GUI 114, and a template management GUI 116. For example, via the course directive GUI 112, one or more users may plan a simulation scan for obtaining anatomical information of a patient as well as plan one or more radiation therapy sessions for delivering radiation therapy to the patient. Additional information about the course directive GUI 112 is presented below with respect to FIGS. 3-7 and 10. Via the multi-patient GUI 114, one or more users may track the progress of RT planning and delivery for a plurality of patients and perform at least some activities related to RT planning via the multi-patient GUI 114. Additional information about the multi-patient GUI is presented below with respect to FIGS. 8 and 9. Additionally, the forms presented to the user(s) for planning simulation scans and radiation therapy sessions may be generated based on templates that may be created and edited via the template management GUI 116. Additional information about the template management GUI is presented below with respect to FIGS. 11 and 12.

The imaging services 130 may include a radiology information system (RIS), a picture archive and communication system (PACS), and/or other computing devices associated with diagnostic imaging, including computing devices associated with and configured to control diagnostic imaging scanners. The computing devices comprising the imaging services 130 may manage scheduling of diagnostic imaging exams, recommend and execute scanning protocols to perform diagnostic imaging exams, process imaging data to generate images and/or volumes, save diagnostic images/volumes in memory, process diagnostic images/volumes to segment anatomical regions of interest, etc. Specifically, the RIS may be configured to track and manage radiology requests and workflow (e.g., CT/x-ray imaging requests and workflows) and the PACS may be configured to store diagnostic images obtained during diagnostic imaging sessions (e.g., images obtained during a simulation scan). The imaging services 130 may receive information from the radiation therapy management system 110, such as a filled simulation scan order form dictating a scan protocol to be used to perform a simulation scan (which may be sent to a medical imaging device such as a CT scanner, for example) and instructions to manage/move diagnostic images (e.g., from a CT scanner to the PACS and/or to other devices for further processing of the diagnostic images).

The RT system 140 may include an oncology information system (OIS) and/or other devices associated with radiation therapy, such as computing devices associated with and configured to control radiation therapy devices (such as linear accelerators). The OIS may be configured to generate and store oncology-based electronic medical records for patients, including cancer screening, diagnoses, treatments, treatment workflows, image reviews, and so forth. The RT system 140 may receive information from the radiation therapy management system 110, such as a filled radiation therapy plan form dictating parameters of a radiation therapy session (which may be sent to a radiation therapy device, such as a computing device controlling a linear accelerator). Additionally, information from the RT system 140 may be retrieved with the radiation therapy management system 110 and used to autofill the simulation order form and radiation therapy plan form.

EMR database 150 may store EMRs for a plurality of patients. An EMR for a patient may include patient demographic information, family medical history, past medical history, lifestyle information, comorbidities (e.g., preexisting medical conditions), current medications, allergies, surgical history, past medical screenings and procedures, past hospitalizations and visits, etc. EMR database 150 may be an external database accessible via a secured hospital interface, or EMR database 150 may be a local database (e.g., housed on a device of the hospital). EMR database 150 may be a database stored in a mass storage device configured to communicate with secure channels (e.g., HTTPS and TLS), and store data in encrypted form. Further, the EMR database is configured to control access to patient electronic medical records such that only authorized healthcare providers may edit and access the electronic medical records. Additionally, information from the EMR database 150 may be retrieved with the radiation therapy management system 110 and used to autofill patient intake information on the course directive GUI and/or multi-patient GUI, the simulation order form, and the radiation therapy plan form. In some examples, various patient information (e.g., identifying information, demographic information) may additionally or alternatively be stored in a hospital information system (HIS), and information from the HIS may be retrieved with the radiation therapy management system 110 and used to autofill the patient intake information, the simulation order form, and the radiation therapy plan form.

While not shown in FIG. 1, one or more workstations may also be communicatively coupled to computing device 102 via network 126. Each workstation may include a processor, memory, communication module, user input device, display (e.g., screen or monitor), and/or other subsystems (similar to the processor, memory, communication module, user input device, and output device of computing device 102) and may be in the form of a desktop computing device, a laptop computing device, a tablet, a smart phone, or other device. Each workstation may be adapted to send and receive encrypted data and display medical information, including medical images in a suitable format such as digital imaging and communications in medicine (DICOM) or other standards. As will be explained in more detail below, the workstations may display graphical user interfaces described herein and may facilitate user interaction with the graphical user interfaces.

The radiation therapy management system 110 may fetch data stored in various data sources, aggregate the data, and share the data with various downstream devices via at least one of multiple connectivity methods. The multiple connectivity methods may include DICOM images, DICOM SRs, HL7, FHIR, proprietary logs associated with a manufacturer or operating system, and so on. Thus, the computing device 102 may be operably coupled to the data sources/downstream devices (e.g., imaging services 130, RT system 140, EMR database 150) via wired connections, wireless connections, and/or any method for communicably connecting systems. As described herein “operably coupled” is to be understood as coupling of elements via connectivity methods (e.g., wired connection, wireless connection, and so on) which enable transfer of data, signals, requests, and/or other information among the operably coupled elements.

It should be understood that computing system 100 shown in FIG. 1 is illustrative and non-limiting, and that another appropriate computing system 100 may include more, fewer, or different components. The various systems included in computing system 100 (e.g., the computing device 102, imaging services 130, RT system 140, and EMR database 150) may be separate devices in communication, as shown in FIG. 1. One or more of the devices described herein may be implemented over a cloud or other computer network. For example, computing device 102 is shown in FIG. 1 as constituting a single entity, but it is to be understood that computing device 102 may be distributed across multiple devices, such as across multiple servers. Further, while radiation therapy management system 110 is shown as being included as part of computing device 102, in some examples, radiation therapy management system 110 may be included as part of one or more other devices, such as implemented as part of imaging services 130 (e.g., in the RIS).

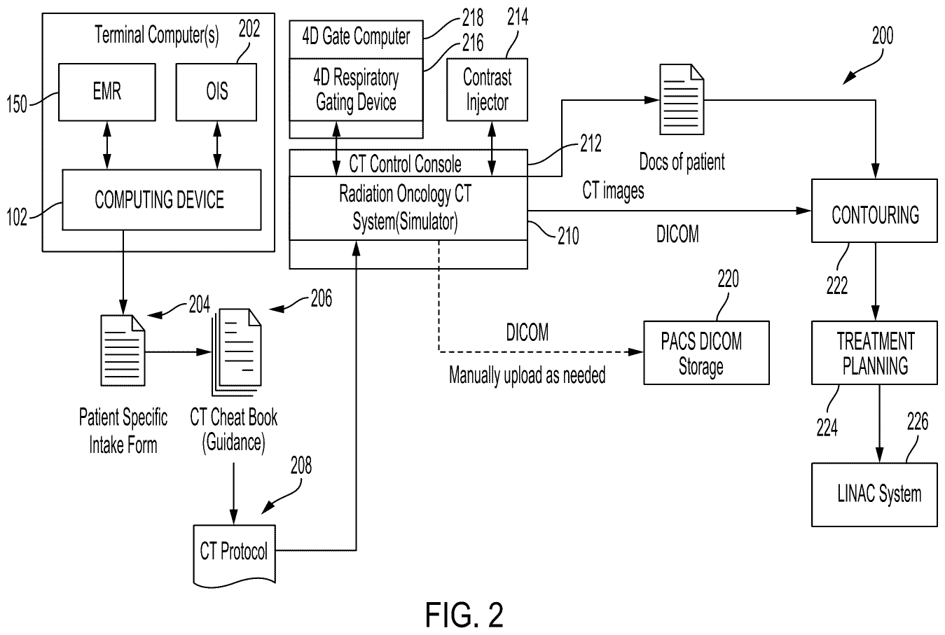

FIG. 2 shows an integrated radiation oncology system 200 that includes aspects of computing system 100. Via the integrated radiation oncology system 200, a patient may be treated with radiation therapy to mitigate cancer, for example. When a patient is treated via the integrated radiation oncology system 200, a workflow may be followed that starts with patient registration. During the registration procedure, a user (e.g., office personnel) may enter patient information to the computing device 102 or another workstation, which sends the patient information to the EMR database 150 and/or an OIS 202 (which as explained above may be implemented on a device that is part of the RT system 140). The patient information may include patient identifying information (e.g., name, date of birth) as well as patient demographic data (e.g., age, gender). The patient information may be gathered with and/or aggregated into a patient-specific intake form 204.

A simulation procedure may be performed on the patient and is planned through the radiation therapy management system 110. For planning the simulation procedure, the course directive GUI 112 may include a simulation order form, which may be part of or separate from the patient-specific intake form 204. A user (e.g., the radiation oncologist) may create the plan for the simulation via the simulation order form. A medical imaging exam such as a CT scan may be performed during the simulation to produce a 3D model of the patient's anatomy for guiding the radiation therapy. As shown in FIG. 2, the information from the patient-specific intake form 204 and/or simulation order form may be used along with CT protocol guidance 206 to generate a CT protocol 208, and a CT scanner 210 may be controlled (by a CT control console 212) according to the CT protocol 208 to perform the CT scan. The CT control console 212 may include a display device configured to display an imaging interface. In some examples, the CT protocol 208 may be displayed via the imaging interface and/or additional information of the simulation scan, as dictated by the information obtained via the course directive GUI, may be displayed via the imaging interface. The imaging interface may be specific to the CT scanner and thus may not display radiation therapy planning information. As such, the imaging interface may have increased display area relative to the course directive GUI available for CT-specific information not displayed via the course directive GUI. In this way, the course directive GUI may display a limited amount of CT-related information (e.g., the simulation order form/summary of the simulation order form) that is relevant to radiation therapy planning.

In addition to the CT scanner 210, the CT scan may be facilitated by a contrast injector 214 (which may inject a contrast agent into the patient prior to the CT scan commencing). Further, a respiratory gating device 216 (controlled by a gate computer 218) may be included to enable respiratory gating of the simulation scan. The images obtained during the simulation scan may be saved in a suitable image archive (e.g., a PACS 220) and sent for further processing to generate the 3D model of the patient's anatomy. The further processing may include contouring performed by a contouring module 222, as shown in FIG. 2. Once the 3D model is available, radiation therapy treatment may be planned using a treatment planning module 224. While a CT scanner is provided herein as an example of a medical imaging device that may be used to facilitate radiation therapy planning and delivery, other medical imaging devices may be used without departing from the scope of this disclosure, such as an MRI scanner, ultrasound device, etc.

Using the radiation therapy management system 110, a radiation oncologist may create a treatment plan for treating the patient that specifies the area of interest for radiation treatment as well as the radiation modality to be used. Specifically, the radiation therapy management system 110 may generate and/or display the course directive GUI 112, which may include a plan intent form. The radiation oncologist may create the radiation treatment plan via the plan intent form of the course directive GUI 112.

For delivery of the radiation therapy, the radiation therapist administers the radiation therapy using a linear accelerator (LINAC) system 226, for example. The course directive GUI 112 may be viewed during administration of the radiation therapy, highlighting the treatment area on a 3D human figure displayed on the GUI. After treatment delivery, regular check-ups are used to keep track of the patient's progress, and the course directive GUI and/or multi-patient GUI may be used to plan any necessary follow-up care.

In the example shown in FIG. 2, the computing device 102, CT control console 212, and gate computer 218 may be located in the same physical location (e.g., within a CT control room, which may be adjacent a room housing the CT scanner). The PACS 220, LINAC system 226, and computing device(s) implementing the contouring module 222 and treatment planning module 224 may be located in a different physical location(s) from the CT control room. Further, it is to be appreciated that the EMR database 150 and OIS 202 may be located at a different physical location(s) than the computing device 102 and that each of the EMR database 150 and the OIS 202 may be implemented with a cloud-based platform and accessed via the computing device 102 or another workstation.

Thus, the course directive GUI and multi-patient GUI show all pertinent data from the OIS and EMR systems during this workflow, including schedule data, simulation order data, demographic data, service data, and data on modality and services. The course directive GUI may present the various pertinent data in a common, human-readable format. The radiation therapy management system may be configured to retrieve the pertinent data from the different data sources (e.g., the OIS and EMR database) in one or more first formats and convert the data to the common, human-readable format. When the simulation order form and plan intent form are filled out, the radiation therapy management system may be configured to convert the simulation order form (or the information obtained via the simulation order form) and/or the plan intent form (or the information obtained via the plan intent form) into one or more second formats (different than the common, human readable format and in some examples, different than the one or more first formats). For example, the simulation order form/information from the simulation order form may be converted to a second format usable by the CT control console/CT scanner. Further, the RT management system 110 may be configured to communicate in various different formats to facilitate performance of the simulation scan based on the information in the simulation order form and image storage and retrieval for CT images, MR images, etc., as well as facilitate performance of contouring and/or segmentation of the images (e.g., to form the 3D model) and radiation therapy delivery based on the 3D model and information in the plan intent form.

The human 3D figure displayed on the course directive GUI highlights the treatment-relevant area and shows the location of any pacemakers, assisting in the delivery of accurate and secure care. Radiation therapists, oncologists, or office personnel, for example, can have the course directive GUI tailored to their individual needs. Additionally, the course directive GUI can be made to be intuitive and user-friendly, with obvious labels and visual cues to aid users in finding the information they require quickly. Overall, this workflow design for radiation oncology can help to improve workflows, efficiency, patient safety, and the overall patient experience.

Turning to FIG. 3, an example of a course directive GUI 300 is shown of a radiation therapy management system, such as the radiation therapy management system 110 of FIG. 2. Course directive GUI 300 may be displayed on a display device (e.g., a display device of a workstation, on a display device of the computing device 102, etc.). Specifically, course directive GUI 300 may be displayed to a radiation oncologist, a radiation therapist, and/or office personnel over the course of radiation therapy planning and administration using the integrated radiation oncology system 200 of FIG. 2. In some examples, the course directive GUI 300 may be launched/displayed in response to a request by a user when the user wishes to commence radiation therapy planning for a patient. Once the radiation therapy for the patient has been planned, the course directive GUI 300 may be launched/displayed by the same user or a different user when the radiation therapy is carried out. In some examples, the user may view the course directive GUI 300 by first logging into a radiation therapy management system, and selecting an RT planning icon, tile, or similar menu listing of one or more options to view the course directive GUI 300. For example, a multi-patient workflow manager GUI (e.g., the multi-patient GUI 114) may be displayed that includes an RT planning icon for each of a plurality of patients scheduled to undergo RT at a particular medical facility or medical network, as shown in FIG. 8 and explained in more detail below. User selection of the RT planning icon may launch the course directive GUI 300. In still other examples, the course directive GUI 300 may be launched from within a different computer system of a hospital, such as an EMR system or an OIS, where the course directive GUI may be listed as a selectable menu option within a UI of the EMR system or OIS. The course directive GUI may facilitate management and planning of RT, and thus may also be referred to herein as an RT management GUI.

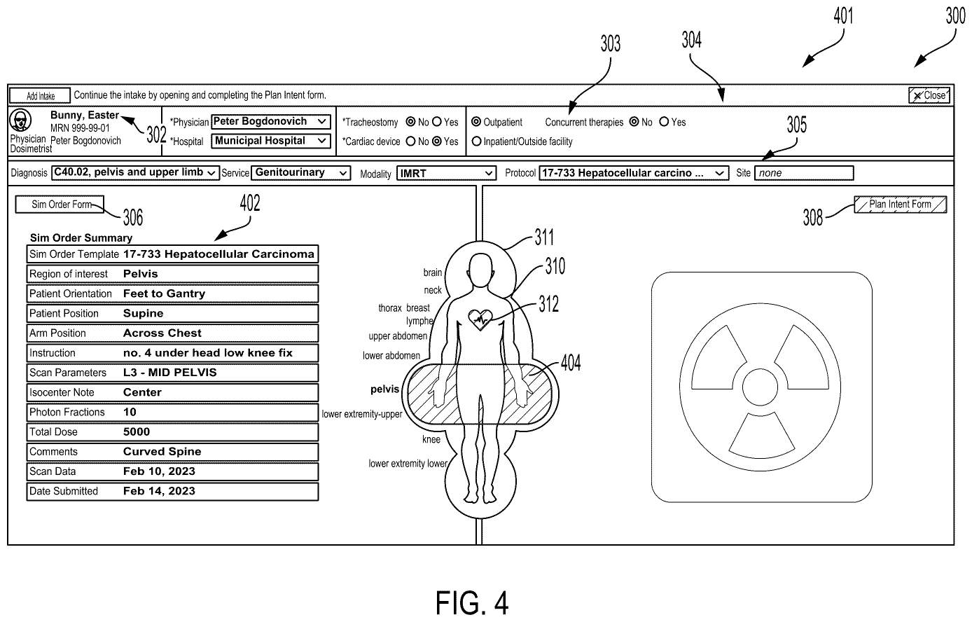

As depicted in FIG. 3, course directive GUI 300 is in first view 301. The course directive GUI 300 may be displayed in different alternative views, examples of which are shown in FIGS. 4-7. Further, the course directive GUI 300 may take on different formats depending on who is viewing the course directive GUI 300. In the example shown in FIGS. 3-7, a physician is viewing the course directive GUI 300 and thus a physician format of the GUI is shown.

The first view 301 may be displayed during patient intake or otherwise when planning of a simulation scan and radiation therapy for a patient is about to be performed (e.g., prior to commencement of the simulation scan and radiation therapy). The GUI 300 may include patient identifying information 302 and an intake information panel 304 whereby information about the patient pertinent to radiation therapy planning may be viewed. The patient identifying information 302 may include the name of the patient and the patient's medical record number (MRN). Once the patient has been identified/selected, information of the patient stored in the EMR database and/or the OIS may be obtained by the radiation therapy management system and used to auto-populate sections of the intake information panel 304, a simulation order form, and a plan intent form, which are explained in more detail below.

The intake information panel 304 may include a first section 303 comprising various display and user input elements, including menus via which a physician administering care to the patient may be viewed/specified and a medical facility at which the patient is being treated may be viewed/specified. Patient information may be indicated via input buttons, such as whether or not the patient has had a tracheostomy, whether or not the patient has a cardiac device (e.g., pacemaker), whether the patient is an outpatient or an inpatient, and whether or not the patient is receiving concurrent therapies. In some examples, at least some of the information displayed as part of the first section 303 may be auto-filled based on patient information stored in the EMR database and/or the OIS.

The intake information panel 304 may further include a second section 305. The second section 305 may comprise user interface elements (e.g., menus) via which the user (e.g., physician) can view/specify the patient's diagnosis, the service being provided, the modality providing the service, the protocol to be followed, and the site at which treatment will be administered. As shown, at least some of the user interface elements may be drop-down menus whereby the user may select from among a predefined list of diagnoses, services, modalities, protocols, and/or sites. In some examples, at least some of the user interface elements may be fields (e.g., text boxes) whereby the user may enter text (e.g., the site may be specified via text entry rather than selection from a drop-down menu). The diagnosis user interface element may allow the user to select/specify the patient's diagnosis (e.g., type of cancer that is to be treated), which may be specified via ICD-10 codes. In some examples, if a diagnosis has already been entered for the patient in the OIS, only that diagnosis may be available for selection via the diagnosis user interface element. The service user interface element may allow the user to specify/select the anatomical region being treated. The modality user interface element may allow the user to specify/select the type of radiation therapy to be administered (e.g., intensity-modulated radiation therapy (IMRT), image-guided radiation therapy (IGRT), stereotactic body radiation therapy (SBRT), stereotactic radiosurgery (SRS)). The protocol user interface element may allow the user to specify/select the protocol to be followed for delivering the radiation therapy, which may include internal protocols identified via the type of cancer being treated or another suitable metric. The site user interface element may allow the user to specify/select the anatomical site being treated.

To facilitate RT planning, the GUI 300 may include a first button 306 that, when selected, launches a first display panel wherein a simulation order form is displayed. The first button 306 may be identified on the GUI 300 as a “Sim Order Form” button. The GUI 300 may also include a second button 308 that, when selected, launches a second display panel, wherein a radiation therapy plan intent form is displayed. The second button 308 may be identified on the GUI 300 as a “Plan Intent Form” button. In the first view 301, the second button 308 may be deemphasized (e.g., displayed in white, gray, or otherwise a different color than the first button 306) to signify that the second button 308 is unavailable for selection, such that the radiation therapy plan intent form cannot be filled out until the simulation order form is filled out. The simulation order form, when displayed in response to selection of the first button 306, may include various menus and fields that the user may interact with (e.g., make selections, enter text) in order to plan a simulation scan for the patient prior to commencement of the radiation therapy. In some examples, the first button 306 may only be selectable once the intake form is complete (e.g., the information in the intake information panel 304 has been filled out).

The GUI 300 further includes a graphical representation of a human, referred to as a human figure 310. The human figure 310 may be a 3D representation of a human, in some examples. When the patient being treated has a cardiac device, the human figure 310 may be modified to include a graphical indication of the device positioned to correspond to where the cardiac device is located in the patient. For example, in FIG. 3, the human figure 310 includes an indication 312 of a pacemaker located in the chest of the patient. The human figure 310 further includes an outline 311 that may convey a 3D nature of the human figure and/or provide additional information, such as indicating the scanning extent of the CT scanner and/or the status of the radiation therapy planning (e.g., the outline may be in gray when planning is ongoing and turn a different color when planning is complete). The human figure 310 may be positioned in a center of the course directive GUI 300, intermediate the first display panel and the second display panel, which may visually emphasize the human figure and ensure that the human figure 310 is referenced during both planning of the simulation scan via the simulation order form and planning of the radiation therapy via the plan intent form.

FIG. 4 shows the GUI 300 in a second view 401. In the second view 401, the simulation order form has been filled out by the user and a summary 402 of the simulation order is displayed in the GUI 300 (e.g., in the first display panel). The summary 402 may include parameters for carrying out the simulation scan, such as region of interest to be scanned, patient orientation and position, a scan protocol to be followed during the scan, and so forth. At least some of the information that is input to the simulation order form and displayed in the summary 402 may be sent to the CT scanner so that the simulation scan may be executed according to the simulation order, and/or the simulation order/summary may be displayed to a scan technologist during the simulation scan. The first button 306 is deemphasized and the second button 308 is emphasized (e.g., displayed with a color to highlight the button) to indicate that the simulation planning is complete and that the radiation planning form is available to be displayed and filled out. Further, highlighting 404 has been placed over the human figure 310 to indicate the region of the patient that is to be scanned during the simulation scan and treated with the radiation therapy, and the region of interest (e.g., pelvis) that is indicated with text is shown in an emphasized manner relative other anatomical regions.

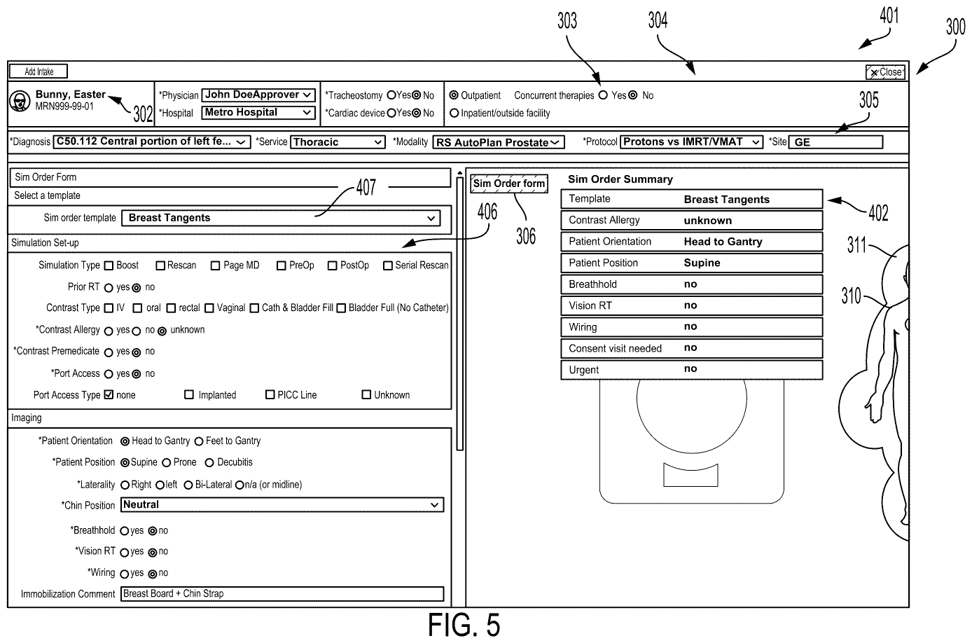

FIG. 5 shows the GUI 300 in the second view 401 with the simulation order form 406 displayed. The simulation order form 406 may include a plurality of fillable fields (e.g., menus, toggle buttons, text boxes) that may be filled by a user to plan the simulation scan. In some examples, the fields included in the simulation order form may be based on a template which may be selected by the user (e.g., via a template menu 407, which in the example shown is a drop-down menu) or auto-selected based on the patient information (e.g., the diagnosis, service, modality, protocol, etc.). Further, in some examples, the fields of the simulation order form may be pre-filled/selected based on the template. Additional details about creating and saving templates for generating simulation order forms are provided below with respect to FIGS. 11, 12, and 15. The user may be able to modify the pre-filled input to the simulation order form if desired. As shown in FIG. 5, the simulation order form 406 may be displayed next to the summary 402, and the summary may be filled as the simulation order form is completed, or the summary may be filled once the simulation order form is completed. In other examples, the summary may be not displayed along with the simulation order form.

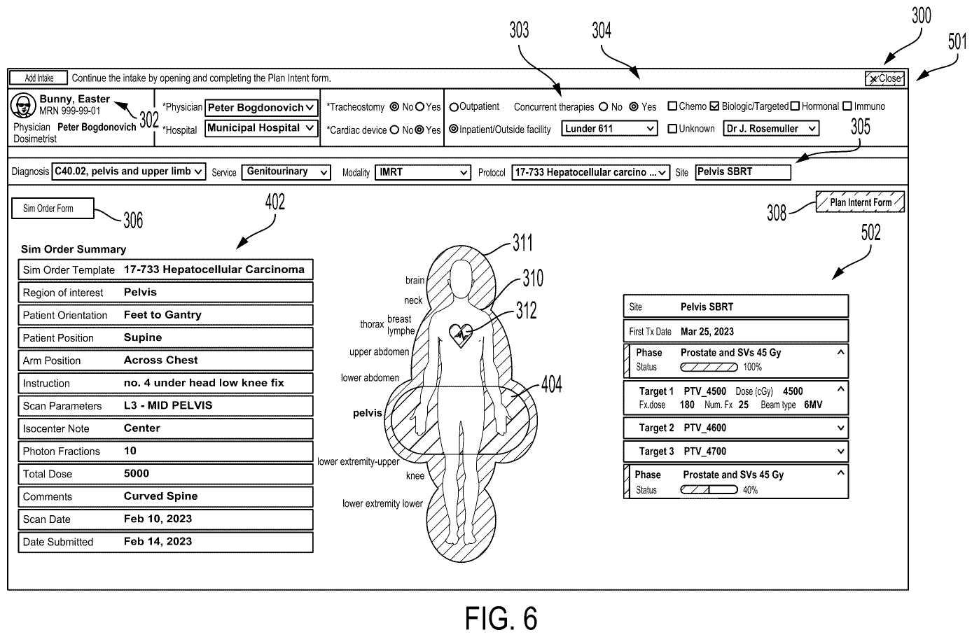

FIG. 6 shows the GUI 300 in a third view 501. In the third view 501, the plan intent form has been or is the process of being filled out by a user and a summary 502 of the plan intent form is displayed in the GUI 300 (e.g., in the second display panel). The summary 502 may include parameters for carrying out the radiation therapy, such as the site for delivery of the radiation therapy, date of first transmission of radiation therapy, various targets for the radiation therapy, and so forth. At least some of the information that is input to the plan intent form and displayed in the summary 502 may be sent to the radiation therapy device (e.g., LINAC system) so that the radiation therapy may be delivered according to the plan intent order, and/or the plan intent order/summary may be displayed to a radiation therapist during the delivery of the radiation therapy. In the third view 501, the summary 502 is displayed along with the summary 402. In this way, the simulation order form button/element and the plan intent form button/element are each selectable to cause the simulation order form and plan intent form, respectively, to be displayed on the course directive GUI. Once filled, the summaries of the simulation order form and the plan intent form are displayed in respective display panels viewable simultaneously on the course directive GUI, wherein the human figure is displayed intermediate the respective display panels.

One or more of the parameters of the plan intent form may be auto-filled based on prior information input by a user about the patient. For example, a user may specify the site for the radiation therapy in the second section 305 of the intake information panel 304, such as “Pelvis SBRT.” The site information may then auto-populate in the plan intent form, filling the site field of the summary 502. Further, some of the fields of the summary 502 may be expanded to view additional information about that field. For example, the summary 502 shown in FIG. 5 includes three targets for receiving radiation therapy (e.g., Target 1, Target 2, and Target 3). Each target has an identifier (e.g., PTV_4500, PTV_4600, and PTV_4700) that may be viewed in the respective target field. When a target field is expanded (e.g., via user selection of a downward-pointed carrot), dose information, beam type, and so forth may be displayed. Additionally, the status of each phase of the radiation therapy may be displayed as a status bar in the respective phase field.

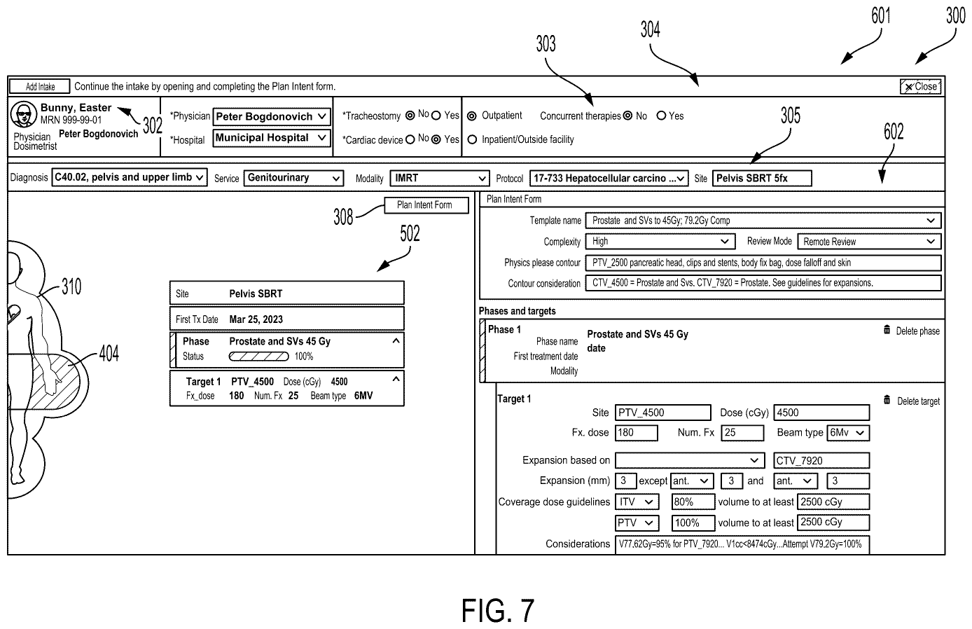

FIG. 7 shows a fourth view 601 of the GUI 300. In the fourth view 601, a portion of the plan intent form 602 is displayed, showing parameters for treating the first target during the first phase of the radiation therapy. Via the plan intent form 602, the user may select/fill various fillable fields to define the radiation therapy that is to be delivered to the patient, add or delete targets and phases, delete structures/organs identified as at risk, add notes, and so forth. If any fields of the plan intent form are auto-populated based on prior information, as described above, the user may review the auto-populated fields and make changes when desired. Similar to the simulation order form, the plan intent form may include a template menu and a user may select a template from a list of predefined templates for the plan intent form, and the fields shown and/or values/information in at least some of the fields may be auto-populated based on the selected template.

Thus, the course directive GUI (such as the GUI 112 and the GUI 300), in concert with the radiation therapy management system, acts to combine information from multiple sources, such the EMR and OIS, into a single graphical user interface (GUI) that presents all the pertinent data in one location. The GUI can give radiation therapists and other medical professionals a visual depiction of the patient's anatomy by combining a human 3D figure with highlighted regions of interest and the position of pacemakers, assisting in the delivery of accurate and safe therapy.

This novel approach to radiation oncology, which combines the integration of multiple systems into a single user interface and the use of a human 3D figure, can boost patient safety, increase treatment accuracy, improve communication and collaboration among healthcare professionals, and enhance the overall patient experience from start of the patient care pathway until creation of treatment plan.

In radiation oncology, real-time synchronization of the patient intake form/simulation order form and the plan intent form has a number of advantages. Radiation oncologists can get the most recent data regarding the patient's demographics, simulation order, modality, and CT schedule by live-syncing the patient intake form with the plan intent form, which may increase patient safety, decrease errors, and improve treatment accuracy.

Additionally, real-time synchronization of the plan intent form and the patient intake form/simulation order form can improve workflow efficiency. Radiation oncologists can quickly and easily access all the pertinent patient data in one location, which makes it simpler to develop a precise and efficient treatment plan.

Additionally, real-time synchronization of the patient intake form/simulation order form and the plan intent form can enhance coordination and cooperation among medical specialists working on the patient's care. By ensuring that everyone is on the same page and working toward the same objective, this can assist to improve the overall standard of care.

The course directive GUI may be modified to match the specific demands of many users, including radiation therapists, oncologists, and office workers, making it a flexible solution that can be adapted to various workflows and environments. Additionally, the course directive GUI is created with clear labels and visual cues to make it easy for users to find the information they seek. Further, the embodiments disclosed herein provide a combination of relative information in that the patient intake form and plan directive have common fields that can be auto filled to make the decision on treatment plan template.

The course directive GUI has been created to display patient demographic, service, modality, simulation order, and schedule data by integrating data from several systems, such as the EMR and OIS. This data can be shown alongside the human figure, giving consumers access to all the necessary data in one location for the radiation oncology patient treatment care pathway.

The human figure can be made interactive so that users can rotate and zoom in on various body sections in order to make the GUI simple to use and intuitive. Clear labels and visual cues can also be included to the user interface to aid users in finding the information they require fast. For example, the example GUI shown in FIGS. 3-7 includes hints/tips, such as “Complete the initial intake by selecting options or providing answers to the questions shown below,” as shown in the first view 301 of FIG. 3 and “Continue the intake by opening and completing the Plan Intent form,” as shown in the second view 401 of FIG. 4.

As explained previously, the course directive GUI may have a part that shows a 3D human figure with a pacemaker placed in a certain spot on the figure. This would make it easier for radiation therapists and other medical practitioners to see where the pacemaker is located and prevent overexposing it to radiation during treatment. The 3D human figure with the treatment-relevant region marked may be displayed in a component of the GUI. In order to ensure that the radiation is given precisely to the appropriate region, this would let radiation therapists and other medical personnel visualize the area that is to be treated.

FIG. 8 shows an example multi-patient workflow manager GUI, referred to herein as a multi-patient GUI 800. The multi-patient GUI 800 is non-limiting example of multi-patient GUI 114 and may present an RT planning workflow for each of a plurality of patients scheduled to receive radiation therapy at a particular location (e.g., hospital) or from a particular hospital system or network. The multi-patient GUI 800 may be displayed (e.g., on a display device of a workstation, on a display device of the computing device 102, etc.) in response to a user request to view the multi-patient GUI 800. The request may be received via user input selecting a multi-patient GUI icon or a menu listing from an interface of the radiation therapy management system. The interface of the radiation therapy management system may include a menu listing one or more applications, and the multi-patient GUI may be reached directly from the menu. The applications listed in the menu may include the EMR database, the OIS, account settings, log-in information, scheduling information for one or more radiation therapy devices, and/or other applications.

The multi-patient GUI 800 may include RT planning information (including an RT planning workflow) for each of the plurality of patients organized into rows, with each row being specific to a patient. For example, a first row 802 shows RT planning information for a first patient. A plurality of additional rows is positioned below the first row, each showing RT planning information for a respective patient of the plurality of patients. The information presented in each row may be organized according to columns, such that a first column 804 depicts patient information (e.g., name, date of birth, medical record ID, and assigned physician, as well as any triggered alerts or notes for that patient), a second column 806 depicts treatment site information, including the course of treatment and current phase of treatment, and a third column 808 depicts treatment modality information (radiation therapy modality, such as volumetric modulated arc therapy (VMAT), IMRT, etc., and treatment room at the hospital).

The columns further include a plurality of workflow columns that indicate each step that is to be taken by one or more clinicians before each patient can receive a scheduled treatment. Each step may include one or more activities. The plurality of workflow columns includes a delivery column 810 that indicates, for each patient of the plurality of patients, a scheduled date that their treatment is to commence. For example, the delivery column 810 indicates, for the first row 802, that the first patient is scheduled to receive treatment starting on Mar. 24, 2024; other patients shown in FIG. 8 may have the same and/or other treatment start dates (e.g., April 1, April 2, etc.). Working backward from the scheduled treatment start date, the timing for when each step/activity of the RT planning workflow that is to be performed prior to the commencement of treatment may be calculated and indicated on the multi-patient GUI 800. The plurality of steps may be organized into categories and depicted along each row in at least a quasi-sequential manner. In the example shown, the categories may include a course directive step, shown in a fourth column 812, an assign activity step, shown in a fifth column 814, a segmentation step, shown in a sixth column 816, a treatment planning step, shown in a seventh column 818, a plan write-up step, shown in an eighth column 820, and a review plan step, shown in a ninth column 822. At least in some examples, the workflow may dictate that the course directive step be performed before the assign activity step is performed, the assign activity step be performed before the segmentation step, and so forth, though some steps may be performed at the same time or in a different order without departing from the scope of this disclosure.

Each step may include one or more activities denoted by an interface element, herein an oval. When an activity has yet to be completed or started (e.g., a future activity), that activity is visually differentiated from activities that have been completed and activities that are partially complete, such as by being shown as an unhighlighted oval of a particular color (e.g., light gray). For example, for the first patient, the first row 802 includes a first plan write-up activity 824 in the eighth column 820 that is shown as an unhighlighted oval to indicate that the first plan write-up activity 824 has not been started (and is not available to be performed). Each step may include one or more activities, with the number of activities shown by the number of ovals in that step. For example, the review plan step may have six activities while the assign activity step may only have one activity.

When an activity has been completed, the activity may be switched to a different color or otherwise visually indicated. Further, when two or more consecutive activities have been completed, the ovals may be joined into a progress bar that is visually indicated by color. For example, in the first row 802, all of the activities for the course directive, assign activity, and segmentation steps have been completed for the first patient. As such, the (previously displayed) ovals are switched to a first progress bar 826 that extends from the first completed activity to the final (e.g., most recent) completed activity and shows that the activities of those steps have been completed. When one or more activities for a given patient is overdue, the progress bar for that patient may be filled with a different color relative to when no activities are overdue. For example, the first progress bar 826 may be filled with a first color (e.g., red) to indicate that at least one activity of the workflow for the first patient is overdue (explained in more detail below). In contrast, a second progress bar 828 for another patient (e.g., shown in the third row from the top) is filled with a second color (e.g., dark gray) to indicate that no activities in the workflow for that patient are overdue.

An available activity for a given patient may be visually indicated via an outline around the oval for that activity. For example, a first available activity 830 for the first patient has an outline around the oval to indicate that activity is the next activity in the workflow that can be completed (e.g., by the user currently viewing the multi-patient GUI 800 or by another user). In the case of the first available activity 830, the activity has been started but not completed, which is shown by half of the oval being filled in with color. Because the first available activity 830 is overdue, the first available activity 830 is outlined in the first color and partially filled with the first color. A second available activity 832 for the other patient is not overdue and is visually indicated with an outline in the second color. The second available activity 832 has yet to be started thus is not filled with the second color. In some examples, more than one activity of the workflow may be determined to be available to be performed, as not all activities have to be performed sequentially. For example, for the patient shown in the second row from the top, three activities are highlighted (via an outline) as being available.

The multi-patient GUI 800 may include a display panel for each available activity that identifies the available activity (e.g., which is the activity that can be completed next), the due date of the activity, and the clinician that has been assigned to complete that activity. For example, for the first available activity 830, a first display panel 834 is included under the first available activity 830. The first display panel 834 indicates the available activity is “TxPlan” (e.g., treatment plan), is assigned to a particular clinician, and is overdue by 5 days. For the second available activity 832, a second display panel 836 is shown that indicates the available activity is “MDPlanRev” (e.g., medical doctor plan review), currently unassigned to a particular clinician, and due in 2 days, 10 hours. In examples where a patient may have more than one available activity (e.g., in the example of the patient shown in the second row from the top), a display panel may be displayed for each available activity, or a display panel may only be shown for one of the available activities.

The multi-patient GUI 800 may also include patient(s) whose RT treatment workflow has been cancelled (e.g., due to the patient deciding not to continue with the RT treatment). For example, the last row in multi-patient GUI 800 includes RT treatment planning information for a patient where the workflow was started but was cancelled before being completed. In such an example, the activities that were completed are shown by a third color (e.g., medium gray) and the point at which the workflow was cancelled is shown with an X over that activity. In some examples, once an RT treatment workflow for a given patient is cancelled, the workflow may no longer be visible in the multi-patient GUI 800 other than when a user specifically requests to view cancelled workflows (e.g., via a filtering function) in order to view historical data, for example.

It is to be appreciated that the view of the multi-patient GUI 800 shown in FIG. 8 is specific for a given clinician (Demouser), such that each patient shown on the multi-patient GUI 800 has an activity in their RT treatment plan/workflow that is assigned to that clinician or could be performed by that clinician. However, other views are possible. For example, various tabs are shown along the top of the multi-patient GUI, and different tabs may be selected to variably filter the patients who are scheduled for RT treatment that are displayed on the multi-patient GUI 800. Similarly, the patients shown on the multi-patient GUI 800 may be filtered via a filter menu. Further, the patients shown may be sorted in a desired manner, such as based on urgency. In examples where more patients are selected to be displayed in the multi-patient GUI 800 than can be accommodated on the screen of the display device, additional patients may be viewed by scrolling (e.g., vertically) or by advancing to another page.

Additionally, a link may be provided on the multi-patient GUI 800 to launch a respective course directive GUI for each patient. For example, the second column 806 includes an icon (e.g., of a human figure) that, when selected, launches a course directly GUI for that patient. As shown, the first row 802 includes a first icon 840. When the first icon 840 is selected, an instance of the course directive GUI (e.g., course directive GUI 300) may be launched that shows information in the course directive GUI that is specific to the first patient. The second column 806 may include other icons that, when selected, cause other actions. For example, user selection of a trash can icon may cause the RT treatment workflow for the associated patient to be cancelled.

In some examples, user selection of certain interface elements of multi-patient GUI 800 may allow some activities of the workflow to be performed directly through multi-patient GUI 800. For example, as explained above, the patient shown in the third row from the top may have multiple available activities highlighted, with a third display panel 838 displayed near one of the available activities. Selection of the third display panel 838 (or selection of the highlighted activity associated with the third display panel 838) may cause display of an activity detail view display panel wherein a user may initiate and/or perform the activity specified by the third display panel 838, which is shown in FIG. 9 and explained in more detail below.

FIG. 9 shows a view 900 of the multi-patient GUI 800 including an activity detail view display panel 902. The activity detail view display panel 902 may be displayed in response to selection of the third display panel 838 and thus includes various display panels and user interface elements to allow a user to view additional information about, and in some examples perform, the activity associated with the selected display panel, which in the example shown is a select CT activity. The activity detail view display panel 902 includes a patient information panel 904 that displays patient information (e.g., date of birth, age, gender, etc.) and an activity status information panel 906 that includes general information about the selected activity, such as whether or not the activity is overdue, the assigned clinician, and due date of the activity, as well as identifying the activity. In the illustrated example, the activity includes selection of CT images/volume of the patient from which a model of the patient's anatomy that is be treated with the RT may be built. Thus, the activity detail view display panel 902 further includes additional display panels via which a selection of CT images/volumes may occur, as well as a notes panel 907 (in which notes about the activity may be input and/or viewed). For example, in input data panel 908, available image files of the patient are listed (e.g., corresponding to imaging exams). For each image file, a view button is displayed. Selection of a view button may launch a view port (e.g., DICOM viewer) in which one or more images stored as part of the selected image file are displayed so that the user may view the images to determine if the selected image file should be used as part of the workflow (e.g., to build the model of the patient's anatomy). Once the user selects an image file, the images/volumes of that file may be transferred to, for example, the contouring module 222 and/or treatment planning module 224. By displaying the activity detail view display panel 902, the user may view images/volumes, select images/volumes, and/or perform other actions directly via the multi-patient GUI and without having to navigate to a separate PACS interface to view the images in each available image file. The activity detail view display panel 902 may further include an output data panel 910, which may also display available output files that may be viewed (e.g., the images selected by the user).

Thus, the multi-patient GUI 800 may visually indicate the progress, available activities, and future activities for an RT treatment planning workflow for each of a plurality of patients. As each activity of a workflow for a patient is completed, the completed activity may be incorporated into a progress bar for that patient that grows as each activity is completed. The current activity that is available to be completed in the workflow is visually highlighted with additional prompting information about the available activity shown in a display panel displayed near the available step. The patients displayed in the multi-patient GUI 800 may be filtered based on clinician. For example, a clinician viewing the multi-patient GUI 800 may filter the multi-patient GUI 800 to view only patients that have outstanding activities assigned to the clinician, or otherwise can be performed by the clinician. In this way, the clinician may view multi-patient GUI 800 to be notified of any overdue, outstanding, and upcoming activities assigned to/performable by the clinician. In some examples, the multi-patient GUI 800 may include a selectable element or menu that, when selected, launches a settings panel wherein a user (e.g., clinician) can set notification preferences for being notified of overdue, current/available, and/or upcoming activities. For example, the settings panel may include selectable elements, menu options, and the like that allow the user to specify to receive notifications of overdue, current/available, and/or upcoming activities via email, SMS, or another suitable method. In some examples, the settings panel may allow the user to specify which type of activity to be notified about, such as only being notified when a particular activity or activities are available to be performed, such as segmentation.

The multi-patient GUI 800 further includes a selectable icon for each patient that, when selected, launches a course directive GUI for that patient, such as the course directive GUI 300 of FIG. 3. In this way, during the course of treatment planning for a patient, a clinician may launch the course directive GUI from the multi-patient GUI so that treatment planning information (e.g., a simulation order form and plan intent form) may be completed and/or viewed for the patient, without forcing the clinician to step through multiple layers of menus to find the simulation order form and plan intent form. Additionally, in some examples, the course directive GUI 300 may be launched in response to user selection of an Add Intake element 842 (e.g., button). For example, selection of the Add Intake element 842 may cause display of an Add Intake panel wherein a user may fill in patient identifying information (e.g., last name and/or MRN). When the name or MRN has a matching record on the OIS, for example, a prompt with the patient's full name may be displayed, and selection of the patient's full name may trigger display of the course directive GUI 300 specific to that patient.

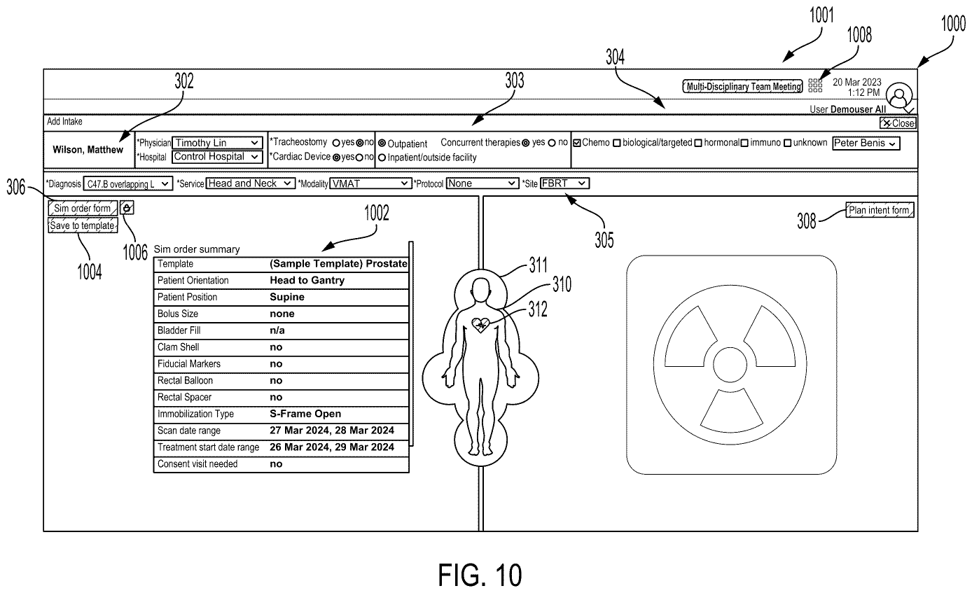

FIG. 10 shows a second example of a course directive GUI 1000 in a second view 1001. The course directive GUI 1000 may be similar to the course directive GUI 300, and thus includes the patient identifying information 302 and the intake information panel 304 (including the first section 303 and the second section 305). Further, the course directive GUI 1000 includes the first button 306, the second button 308, the human figure 310, the indication 312, and the outline 311. The description provided above for the patient identifying information 302, the intake information panel 304 (including the first section 303 and the second section 305), the first button 306, the second button 308, the human figure 310, the indication 312, and the outline 311 likewise applies to the course directive GUI 1000.

The course directive GUI 1000 shown in FIG. 10 is shown in the second view 1001, such that a summary 1002 of the information input to the simulation order form is displayed, similar to the summary 402. Further, the course directive GUI 1000 includes a third button 1004 that, when selected, saves the information entered to the simulation order form as a template, in a template library. As will be explained below, a user may select a saved template to auto-populate the information in the simulation order form. The third button 1004 may be identified on the GUI 1000 as a “Save to template” button. Selection of the third button 1004 may automatically save the filled simulation order form as a template and/or launch display of a template management GUI, which is shown in FIG. 11 and explained in more detail below, via which the filled simulation order form may be viewed and further edited before being saved as a template. By automatically saving a filled simulation order form as a template, creation of the template from scratch may be avoided. By avoiding creating the template from scratch, the performance of one or more devices of the RT management system may be improved by lowering the likelihood erroneous templates may be created. Further, network bandwidth may be lowered by avoiding the presentation of the form designer panel of the template management GUI (described in more detail below) and lowering data transmission between the device displaying the template management GUI and a database of the RT management system storing the saved templates.

The GUI 1000 further includes a fourth button 1006 that, when selected, launches a print display panel. Via the print display panel, a user may view a preview of the summary 1002 of the simulation order form, select print settings, and command the summary 1002 be printed at a selected printer. GUI 1000 further includes a system menu 1008. When selected (e.g., hovered over), the system menu 1008 displays options for switching the GUI displayed on the display device. For example, the system menu 1008 may include a listing of “Workflow Manager,” “Protocol Management,” and “Template Management.” Selection of the “Workflow Manager” may prompt display of the multi-patient GUI 800. Selection of the “Template Management” may prompt display of the template management GUI, which is shown in FIG. 11 and explained in more detail below. Selection of the “Protocol Management” may prompt display of a protocol management GUI, wherein a clinical trial protocol list may be managed (e.g., clinical trial protocols may be added, edited, and/or deleted). It is to be appreciated that both GUI 300 and multi-patient GUI 800 includes a system menu similar to system menu 1008 to allow a user to navigate among the various GUIs disclosed herein. Further, once a user has filled out the plan intent form, a button similar to third button 1004 may be displayed to allow the user to save the plan intent form as a template, as well as a button similar to fourth button 1006 to allow the user to print the plan intent form.

FIG. 11 shows a template management GUI 1100 in a first view 1101. The template management GUI 1100 is a non-limiting example of template management GUI 116 and includes a side bar with multiple menu options, including a template management menu 1102. In response to a user input (e.g., selecting the template management menu 1102 or toggling the carrot in the template management menu 1102), a list of selectable options may be displayed related to template management, including “Sim Order Template,” “Plan Intent Template,” and “Intake Template.” In the first view 1101, the “Sim Order Template” option has been selected, launching display of a simulation order template menu panel 1104 that lists available/stored simulation order templates. It is to be appreciated that user selection of the “Plan Intent Template” option may trigger display of a plan intent template menu panel where available/stored plan intent templates may be viewed, modified, and/or generated. Similarly, user selection of the “Intake Template” option may trigger display of an intake template menu panel where available/stored intake templates may be viewed and modified.

Via the simulation order template menu panel 1104, previously generated and stored simulation order templates may be listed (e.g., by name) and optionally with additional information about each template. For example, the simulation order template menu panel 1104 may include a list 1106 of each simulation order template in rows, with identifying information displayed in columns. The identifying information for each template may include Name, Hospital, Protocol, Service, Modality, Comments, Last Modified, Active, Review, and Operate. The Active column may include a toggle button for each template that a user may select to activate, or deactivate, that template. Only activated templates may be available for selection when filling a simulation order form. The Review column may indicate whether or not that template has been reviewed by an administrator, a lead clinician, or another entity. To view or modify/edit an existing template, a user may select a desired template from the list 1106 of templates, which may launch a second view of the simulation order template menu panel (shown in FIG. 12). For example, the Operate column may include a kebab menu for each template. Selection of a kebab menu for a template may cause a pop-up display panel to be displayed that lists a menu of selectable options, including edit, preview, copy, delete, and version history. Selection of the edit option from the menu may launch the second view shown in FIG. 12. If the user selects the delete option, that template may be deleted. If the user selects the copy option, the template may be copied and the user can then adjust desired attributes/setting of the copied template. If the user selects the version history option, the differences between the last saved version and the current version of the template may be viewed. If a user wishes to generate a new template, the user may select a new template element 1108, which is depicted in FIG. 11 as a “Create New Template” button. Selection of the new template element 1108 may launch a view that is similar to the view shown in FIG. 12, but without any fields of the simulation order form pre-filled and/or with at least some default fields included on the form.

FIG. 12 shows a second view 1201 of the template management GUI. In the second view 1201 shown in FIG. 12, a previously-generated template is displayed to allow a user to edit/modify the template. The second view 1201 includes a form designer panel 1202 that includes a plurality of questions organized into sections, with each question including at one or more fillable/selectable fields, options, or menus. The sections shown in FIG. 12 include simulation set-up and imaging, and additional sections may be viewed by scrolling (e.g., vertically) or advancing to the next page. The specific questions included a given template may be user-specified and can be modified via a question bank 1220. For example, selecting a question (e.g., orientation, contrast) from the question bank 1220 causes that question to be added to the bottom of the form designer panel 1202. The questions in the question bank 1220 may be predefined and may be adjusted via a tool bar 1230, which is explained in more detail below.

The options/fields listed for each question may be defined via an options bar 1210. The options bar 1210 may identify a selected question (e.g., via the Field Name section) and options displayed for the selected question, with the ability to set an option as a default, remove an option, and add an option. As an example, in the simulation set-up section of the form designer panel 1202, a plurality of questions is displayed for setting various parameters for initializing/setting up the simulation scan, such as a simulation type, whether or not the patient has undergone prior RT, contrast type, whether or not the contrast should be premedicated, whether or not to use port access, and port access type. The simulation type question, as an example, may include a predefined list of types of simulation scans (e.g., boost, rescan, pre-op, post-op), and the user may add another type of simulation to the predefined list via the options bar 1210. For example, when the user selects (e.g., hovers over) the simulation type field of the form designer panel 1202, the information shown in the options bar 1210 may be customized to that field; as shown, the options bar 1210 depicts “Simulation Type” as the field name and includes the pre-defined list of simulation types with selection boxes (to make that simulation type the default setting of the template) and deselection crosses (to remove that simulation type from the form designer panel 1202). Further, the options bar 1210 includes a “New Option” element that, when selected, allows the user to define a new option for that question (e.g., a new simulation type). Further, the options bar 1210 allows a user to make the question mandatory (or not) and make the question visible or hidden.

In the imaging section of the form designer panel 1202, a plurality of questions is displayed for setting various parameters for imaging during the simulation scan, such as patient orientation, patient position, laterality, chin position, breathhold, vision RT, wiring, arm position, comments about immobilization, and so forth. While not shown in FIG. 12, in the form designer panel 1202, the user may also be able to specify the imaging technique/scan parameters, isocenter, dose, and/or other imaging parameters.

As explained above, new sections/questions may be added to the template via the question bank 1220. When a user wishes to add a section and/or question that is not currently available via the question bank 1220 (e.g., a custom question), the user may add the section or question via the tool bar 1230. For example, to add a new section, the user may select the “Section” button of the tool bar 1230, which may launch a dialog box that includes a field for specifying a name of the new section. The new section may then be added to the bottom of the form designer panel 1202. To add questions to the new section, the user may select the type of question to be added from the tool bar 1230. For example, if the question will only accept one option/answer, the “Single select” button may be selected; if the question will accept more than one option/answer, the “Multiple select” button may be selected. Other types of questions include text (e.g., a fillable text box), number, and description. The questions and options may initially be generic; when a new question is added, the question may be listed on the form designer panel 1202 as “Question 1” and each option may be listed as “Option 1”, “Option 2,” etc. The user may then customize the name of the question and the description of the options via the options bar 1210 as explained above.

For example, if the user wishes to customize the template to include a “Gating” question with the option of selecting yes or no, the user may add a new section or add a new question to an existing section, and select the “Single select” button from the tool bar 1230. Then, via the options bar 1210, the user may edit the field name to “Gating” and edit the options so that the options are yes and no.

As appreciated from FIG. 12, the options bar 1210 includes a selectable link called “Show Hidden Rules.” When selected, the “Show Hidden Rules” link launches a display rules panel via which a user may set rules for compounded questions. A compounded question may include a question that follows a specific answer to a previous question. For example, in the example above, if the user selects “yes” to the question of gating, a second question (e.g., asking the type of gating to be performed) may be displayed, and the second question may not be displayed when the user selects “no” to the question of gating. The display rules panel may include a first drop-down menu of the options specified for the selected question (e.g., yes and no). When a user selects an option (e.g., yes), a second drop-down menu may become selectable, with other predefined questions available from the second drop-down menu. The user may select the question (e.g., the second question of gating type) that is to be triggered when the specified option (e.g., yes) to the first question is selected.

The second view 1201 additionally includes a save form element 1242, a preview element 1244, and a cancel element 1246, as well as a back button 1248. When the save form element 1242 is selected, the information specified via the form designer panel 1202 may be saved (e.g., as a template). When the preview element 1244 is selected, the currently-filled simulation order template may be viewed (e.g., as a summary). When the cancel element 1246 element is selected, all information entered but not saved will be discarded and the previous view of the GUI displayed (e.g., the first view 1101); similarly, selection of the back button 1248 may revert back to the first view 1101.

Thus, the template management GUI allows for creation of fully-customized simulation order templates. The simulation order templates described herein may be customized for the questions asked via the template (e.g., simulation type, contrast type, patient orientation), the answers available for each question, and default selections for at least some of the answers. Multiple different types of questions can be included in a template, such as single select, multiple select, text, number, and description, as well as compounded questions. The template management GUI may present a series of menus that enable a user to add new sections, questions, and answers to a template. As templates are built, questions added to the templates may be saved in a question bank so that common/frequently used questions may be visualized and easily selected, avoiding users having to create all questions from scratch each time a template is created or edited. The ability to fully customize the templates may provide multiple advantages. For example, different simulation scans for different RT delivery modes, anatomies, etc., may dictate different parameters be set for the simulation scans. As new RT protocols are developed, older templates may become obsolete and/or a new template may be demanded with questions/answers not previously anticipated. Further, simulation scans may be performed on a variety of scanners from different manufacturers and customizing templates may allow simulation orders to be created for CT scanners, MR scanners, etc., dictating parameters for specific scanners. The customization of the templates may allow different medical facilities to use preferred terminology and ensure facility-specific simulation scan protocols are followed.

The templates disclosed herein may be saved in non-relational databases as data objects. Non-relational databases may allow for storage of unstructured data, as opposed to relational databases that demand the stored data be structured. Thus, the use of relational databases to store the templates may allow for the customization discussed above, as virtually any question with any number and type of answers may be saved as part of a template, and the data stored in the non-relational database may be retrieved when a template is selected. Data retrieval via the non-relational database may be fast owing to a search being performed on a single data structure instead of having to query multiple tables as in a relational database. Further, templates created and for a specific medical facility may be stored in a non-relational database locally (e.g., on a device located at that medical facility rather than stored via the RT management system cloud-based system). By storing templates in non-relational databases locally, data retrieval speed may be increased by avoiding having to query all templates saved for all medical facilities and allows saved templates to be easily deleted.

Referring back to FIG. 11, the template management menu 1102 allows a user to select a Plan Intent Template option, which causes a template GUI to be launched for managing (e.g., editing, deleting, adding) plan intent form templates. The template GUI for managing plan intent form templates may include fillable fields for specifying targets, phases, and guidelines for the plan intent form. The template GUI for managing plan intent form templates may be substantially similar to the plan intent form displayed as part of the course directive GUI (e.g., as shown in FIG. 7) and may not include customizable questions as described above for the simulation templates.

An example workflow creation and progression for a patient is presented below. When a new patient is scheduled for RT treatment, the patient may be added to the multi-patient GUI 800 by selecting the Add intake element 842 and keying in the patient name or MRN. Once the patient intake is completed, the simulation order form for the patient may be filled out via the course directive GUI (e.g., GUI 300 or GUI 1000). After successful completion of the patient intake form and simulation order form, a patient workflow for the patient is created, which can be seen on the multi-patient GUI. For the newly-created workflow, the first activity is shown as completed, indicating that the patient intake activity has been completed successfully. Patient demographic information may be sent to an imaging modality (e.g., CT scanner) automatically. Based on the scan dates provided in the simulation order form, as the imaging scan (e.g. CT scan) is completed, the progress may be viewed on the multi-patient GUI (e.g., the second activity may progress to completed). The next activity may then be enabled, which is to view the images (e.g., the CT images) by selecting the “SelectCT” on the multi-patient GUI 800 (e.g., as shown on the third display panel 838). When the “SelectCT” is selected, CT images of the patient are automatically loaded for review. As explained above, the activity detail view display panel 902 may be displayed that includes an input data panel 908; selection of one of the view buttons of the input data panel 908 causes the CT images to be displayed (e.g., via a DICOM viewer). If the user is satisfied with the CT images, the user may select a “Start” button of the activity detail view display panel 902 to start the activity, which in this example is to send selected CT images for review by a different user. To select the images, the user may select the corresponding image check box and select “Transfer” from the activity detail view display panel 902 to send the image to the next activity. When the images have all been selected, the user may select “Finish” from the activity detail view display panel 902.

A different user will be able to start the next activity to review the CT images again. To review, the (different) user may select “ApproveCT” displayed in a display panel associated with the activity on the multi-patient GUI 800. An activity detail view display panel is then displayed via the multi-patient GUI that allows the user to review the selected CT images. If the user approves the CT images, the user may select the “Approve” button and optionally add any approval notes. The approve activity is then set to complete in the progress bar for the patient on the multi-patient GUI. The user may then assign the next activity to a clinician, which is an Intent activity, via an activity detail view display panel. The assigned user may then be able to finish the Intent activity. Is to be appreciated that each of the activities described above (e.g., patient intake, simulation order form, perform CT scan, view CT images, review CT images, and intent) are included as part of the “course directive” step of the workflow. It is to be appreciated that further activities may be performed as above. For example, each activity may be assigned a user to complete the activity, and when a given user logs in to view the multi-patient GUI, the user may view all of their assigned activities and complete the activities via the multi-patient GUI. In some examples, different interfaces/viewports may be launched to complete the activity, such as segmentation.

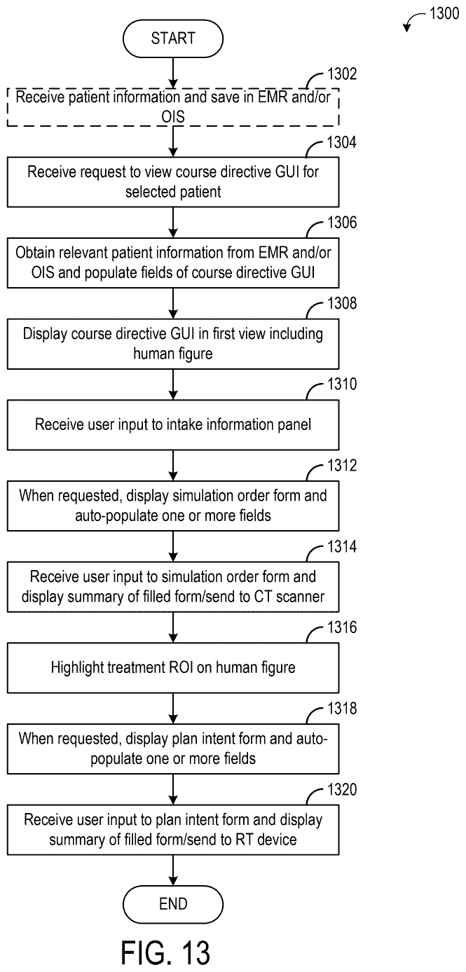

Turning now to FIG. 13, a flowchart illustrating a method 1300 for integrating radiation therapy data and displaying the integrated data via a course directive GUI is shown. Method 1300 may be executed according to instructions stored in memory of a radiation therapy management system of a computing device and executed by one or more processors of the computing device, such as computing device 102 of FIG. 1.

At 1302, method 1300 includes receiving patient information for a selected patient and saving the patient information in an EMR database and/or an OIS. The patient information may include patient name and demographic information including date of birth, sex, etc. The patient information may further include the presence of any cardiac devices or other implants. The patient information may be received via the computing device executing method 1300 (e.g., during patient intake prior to initiation of the radiation therapy), or by a different computing device (e.g., a workstation) in communication with the EMR database and/or the OIS. In either case, the patient information may be saved in the EMR database and/or the OIS, such as the EMR database 150 of FIG. 1 and the OIS 202 of FIG. 2.

At 1304, a request is received to view a course directive GUI for the selected patient. The request may be received via user input selecting a course directive GUI icon or menu listing from an interface of the radiation therapy management system. The interface of the radiation therapy management system may include a menu listing one or more applications, and the course directive GUI may be reached directly from the menu. The applications listed in the menu may include the EMR database, the OIS, account settings, log-in information, scheduling information for one or more radiation therapy devices, and/or other applications. The selected patient may be specified via user input (e.g., the user requesting to view the course directive GUI may enter or select the patient's name or medical record number). In other examples, the course directive GUI may be reached via a multi-patient workflow manager GUI (e.g., as shown in FIG. 8). At 1306, relevant patient information of the selected patient is obtained from the EMR database and/or the OIS and one or more fields/user interface elements of the course directive GUI are populated with the relevant patient information. The course directive GUI is then displayed in a first view, as indicated at 1308. In the first view, a patient identification section and/or an intake information panel may be populated with the relevant patient information. The first view may further include a human figure, as shown in FIG. 3. If the patient information indicates that the selected patient has a pacemaker or other cardiac device, the presence of the cardiac device may be graphically indicated on the human figure.

At 1310, method 1300 includes receiving user input to the intake information panel of the course directive GUI. For example, the user may enter input to fill any fields of the intake information panel not auto-populated, such as selecting a patient diagnosis, service, modality, protocol, and/or site, or adjusting any auto-populated fields that include inaccurate information.

At 1312, when requested, a simulation order form may be displayed via the course directive GUI. The simulation order form may be displayed in in a first display panel of the GUI in response to user input to the course directive GUI, such as user selection of a button/interface element identifying the simulation order form (e.g., the first button of FIG. 3). As explained previously, the simulation order form may be filled out in order to specify how a simulation scan on the selected patient is to be conducted, and thus includes fields such as the region of interest to be scanned, patient position during the scan, x-ray dose of the scan, and so forth, as shown in FIG. 5. In some examples, one or more fields of the simulation order form may be auto-populated based on patient information and/or based on a selected template. For example, the user may specify a radiation therapy protocol to be followed for delivery of the radiation therapy, via selection from a drop-down menu of the course directive GUI (e.g., in the intake information panel). The simulation order form may be selected and/or auto-populated based on the selected protocol. In other examples, the simulation order form may include a template menu which may display saved simulation templates, and user selection of a template from the template menu may cause specific fields (e.g., questions) to be displayed as part of the simulation order form and may also cause auto-population of one or more fields of the simulation order form. When a template is selected, the template may be retrieved from a local non-relational database (e.g., the data objects saved as the template in the database may be retrieved) and used to create the simulation order form that is displayed on the course directive GUI. At 1314, user input may be received to the simulation order form, in order to fill out the simulation order form. The user may evaluate the auto-populated field(s) and make any changes, if desired, as well as fill any unfilled fields. Once the simulation order form is complete, a summary of the simulation order form may be displayed in the first display panel on the course directive GUI, as shown in FIG. 4. Additionally, the simulation order form/information entered via the simulation order form may be sent to a CT scanner (e.g., the CT control console of the CT scanner) or otherwise used to generate a CT scan protocol that is sent to the CT scanner for carrying out the simulation scan of the patient.

At 1316, a treatment region of interest (ROI) is highlighted on the human figure of the course directive GUI. Once the treatment ROI is defined (e.g., via the simulation order form or via other user input), the treatment ROI may be highlighted on the human figure (e.g., as shown in FIG. 4) to provide additional visual cues to the user when planning the radiation therapy, to help avoid inaccurate targeting of the radiation therapy (e.g., avoid directing the radiation therapy to the pacemaker of the patient).

At 1318, when requested, a plan intent form is displayed via the course directive GUI. The plan intent form may be displayed in a second display panel of the GUI in response to user input to the course directive GUI, such as user selection of a button/interface element identifying the plan intent form (e.g., the second button of FIG. 4). As explained previously, the plan intent form may be filled out in order to specify how radiation therapy on the selected patient is to be delivered, and thus includes fields such as the region of interest to be treated, phases, targets for each phase, dose for each target and phase, and so forth, as shown in FIG. 7. In some examples, one or more fields of the plan intent form may be auto-populated based on patient information and/or information entered to the simulation order form, such as the region of interest to be treated. Additionally or alternatively, the fields included in the plan intent form may be selected based on a selected template and one or more fields of the plan intent form may be auto-populated based on the selected template (e.g., selected from a template menu of the plan intent form).

At 1320, user input to the plan intent form is received and, once the plan intent form is complete, a summary of the plan intent is displayed in the second display panel of the course directive GUI, as shown in FIG. 6, for example. Further, the plan intent form/information entered via the plan intent form may be sent to a radiation therapy device, such as a LINAC system, so that the LINAC system can be controlled according to the radiation therapy plan specified via the plan intent form. It is to be appreciated that the user that enters the user input to fill the plan intent form may be the same user or a different user than the user that entered the user input to fill the simulation order form. Further, when the course directive GUI is displayed during the simulation scan and/or delivery of radiation therapy, different users may view the course directive GUI than filled out the forms of the course directive GUI. For example, a scan technologist may view the course directive GUI during execution of the simulation scan while a radiation technologist may view the course directive GUI during delivery of the radiation therapy.

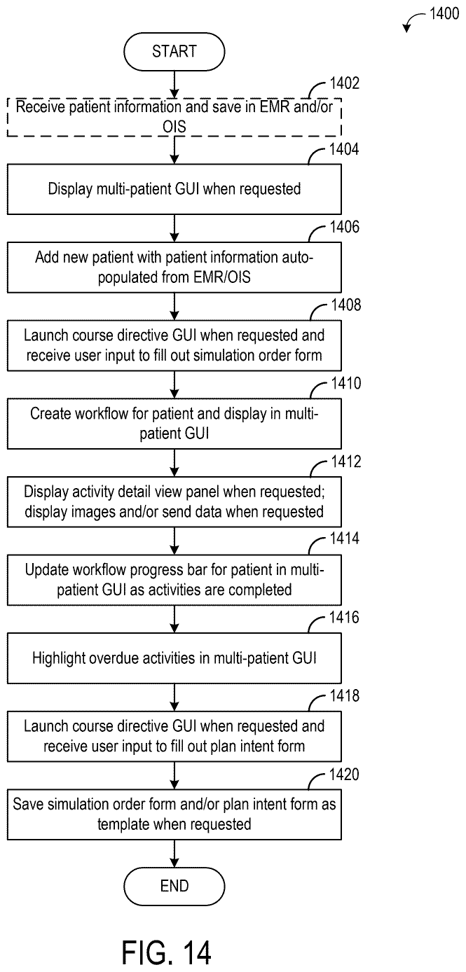

FIG. 14 shows a flowchart illustrating a method 1400 for managing a patient workflow via a multi-patient GUI (such as multi-patient GUI 800). Method 1400 may be executed according to instructions stored in memory of a radiation therapy management system of a computing device and executed by one or more processors of the computing device, such as computing device 102 of FIG. 1. At 1402, method 1400 optionally includes receiving patient information for a selected patient and saving the patient information in an EMR database and/or an OIS. The patient information may include patient name and demographic information including date of birth, sex, etc. The patient information may further include the presence of any cardiac devices or other implants. The patient information may be received via the computing device executing method 1400 (e.g., during patient intake prior to initiation of the radiation therapy), or by a different computing device (e.g., a workstation) in communication with the EMR database and/or the OIS. In either case, the patient information may be saved in the EMR database and/or the OIS, such as the EMR database 150 of FIG. 1 and the OIS 202 of FIG. 2.

At 1404, a multi-patient GUI is displayed when requested. The request may be received via user input selecting a multi-patient GUI icon or menu listing from an interface of the radiation therapy management system. For example, after user authentication, a user may be presented the multi-patient GUI as a default. In other examples, the user may view the multi-patient GUI in response to selecting the multi-patient GUI from a menu, such as the system menu (e.g., system menu 1008 of FIG. 10) displayed on the course directive GUI or template management GUI. In this example, the interface of the radiation therapy management system may include a menu listing one or more applications, and the course directive GUI may be reached directly from the menu.

At 1406, a new patient may be added to the multi-patient GUI and patient information may be auto-populated into the multi-patient GUI from the EMR and/or OIS. For example, a user may enter a relatively small amount of patient identifying information (e.g., last name, MRN) and then select the patient from a menu that presents the patient's full name in response to the patient identifying information. In response, relevant patient information of the selected patient is obtained from the EMR database and/or the OIS and one or more fields/user interface elements of the multi-patient GUI are populated with the relevant patient information. It is to be appreciated that, in response to a request to add a new patient, the multi-patient GUI may include a panel displaying patient information and intake information, similar to patient identifying information 302 and intake information panel 304 of the course directive GUI, and some fields of the panel may be auto-filled based on the information from the EMR and/or OIS.

At 1408, the course directive GUI is launched when requested, and user input is received to fill out a simulation order form. The course directive GUI may be launched as explained above (e.g., in response to user selection of an icon, such as first icon 840, on the multi-patient GUI). Further, the simulation order form may be displayed via the course directive GUI and the user input may be received in order to fill out the simulation order form as explained above with respect to FIG. 13. At 1410, a workflow is created for the new patient and displayed in the multi-patient GUI. As explained above with respect to FIG. 8, the workflow may be visualized with an oval for each activity of the workflow yet to be performed, and a progress bar for each completed activity. Further, for each activity that is available to be performed, a display panel may be displayed identifying the activity and due date of the activity, for example, and selection of a display panel may launch an activity detail view panel via which the associated activity may be performed.

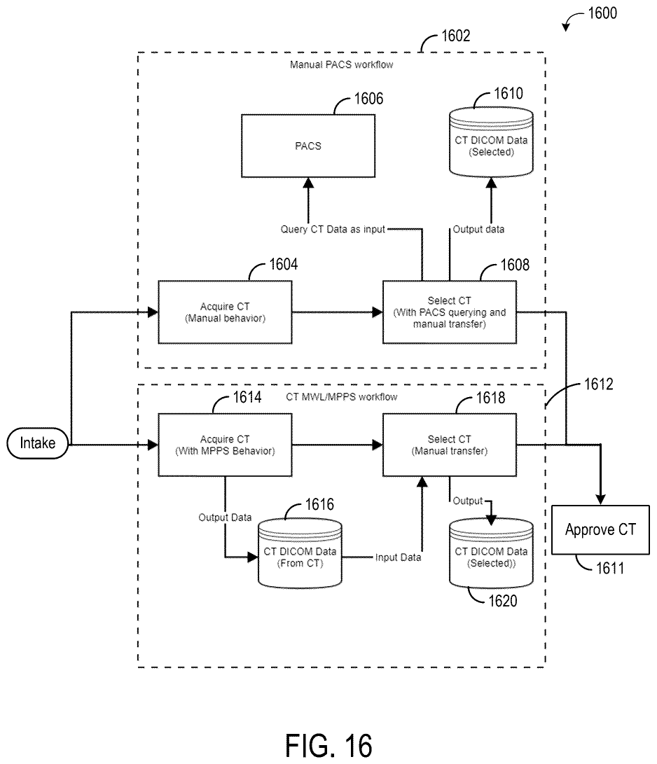

Thus, at 1412, an activity detail view panel is displayed when requested. As explained above with respect to FIG. 9, the activity detail view panel may enable the user to perform an activity, such as reviewing and/or selecting CT images (e.g., from the simulation scan). The activity detail view panel may list available images and include various user interface elements that may be selected to view the images, select certain images, transfer selected images, and so forth. Thus, method 1400 may further include displaying images and/or sending data (e.g., images) when requested (e.g., in response to user input via the activity detail view panel). Specifically, based on the user input to an activity in a workflow (e.g., selecting a display panel associated with an activity, such as “SelectCT”), the patient whose images will be viewed is identified. The radiation therapy management system may communicate with an image archive storing the images (e.g., the PACS) to identify the pertinent images for the patient (e.g., identify the images obtained during the simulation scan), and display a list of the pertinent images in the activity detail view display panel (e.g., by exam or each image individually). When the user enters input requesting to view the images, an image viewer (e.g., DICOM viewer) may be launched automatically to enable the images to be viewed and selected. Further, the selected images may be automatically sent for subsequent analysis, which may include sending to a separate device for performing segmentation, for example. In doing so, the user can directly view only the pertinent images without having to navigate to an interface of the image archive, search for the pertinent images, extract selected images, and send the selected images for subsequent analysis (e.g., additional review, segmentation, etc.). The radiation therapy management system may orchestrate the DICOM-based image search and image move, which may allow efficient data transfer among devices (e.g., the PACS, the device executing the segmentation module) and avoid erroneous searches, image display, etc., that may accompany the user executing the image search, image move, etc., via the image archive directly. Additional information about automatically viewing and selecting images via the multi-patient GUI is presented below with respect to FIG. 16.

At 1414, the workflow progress bar for the patient in the multi-patient GUI is updated as each activity of the workflow is completed. Thus, the progress bar may grow across the GUI as subsequent activities are completed. Further, as indicated at 1416, any overdue activities (e.g., activities that have not been completed by their due date) may be highlighted on the multi-patient GUI. For example, the ovals for overdue activities may be visualized in a different color than ovals for activities that are not overdue. Further, the progress bar may be visualized in a different color when the workflow for the patient has any overdue activities.

At 1418, method 1400 includes launching the course directive GUI when requested and receiving user input to fill out a plan intent form. The course directive GUI may be launched as explained above (e.g., in response to user selection of an icon, such as first icon 840, on the multi-patient GUI). Further, the plan intent form may be displayed via the course directive GUI and the user input may be received in order to fill out the plan intent form as explained above with respect to FIG. 13. At 1420, method 1400 may include saving the simulation order form and/or plan intent form as a template when requested. As explained above with respect to FIG. 10, the course directive GUI may include buttons that are displayed when the simulation order form has been completed and when the plan intent form has been completed. Selection of the button associated with the simulation order form may cause the filled out simulation order form to be saved as a template in a template library, so that subsequent simulation order forms may be pre-filled with the information in the currently-filled simulation order form, when desired. Similarly, selection of the button associated with the plan intent form may cause the filled out plan intent form to be saved as a template in a template library, so that subsequent plan intent forms may be pre-filled with the information in the currently-filled plan intent form, when desired.

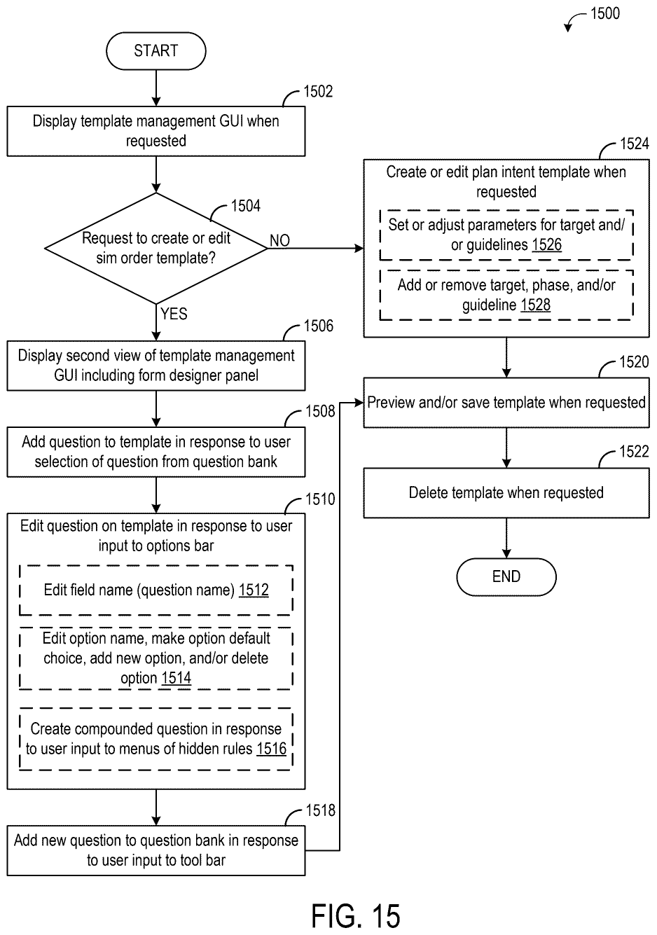

FIG. 15 shows a flowchart illustrating a method 1500 for managing templates via a template management GUI (such as template management GUI 1100). Method 1500 may be executed according to instructions stored in memory of a radiation therapy management system of a computing device and executed by one or more processors of the computing device, such as computing device 102 of FIG. 1.

At 1502, a template management GUI is displayed when requested. The request may be received via user input selecting a template management GUI icon or menu listing from an interface of the radiation therapy management system. For example, a user may view the template management GUI in response to selecting the template management GUI from a menu, such as the system menu (e.g., system menu 1008 of FIG. 10) displayed on the course directive GUI or multi-patient GUI. In this example, the interface of the radiation therapy management system may include a menu listing one or more applications, and the template management GUI may be reached directly from the menu.

At 1504, method 1500 determines if a request to create or edit a simulation order template has been received. The template management GUI may include a side bar/side menu that lists the various template types that may be viewed and edited, including simulation order templates and plan intent templates. Responsive to user selection of the simulation order templates from the menu, a template library of saved simulation order templates may be displayed and a “Create New Template” button may also be displayed. User selection of the “Create New Template” button may launch a second view of the template management GUI that includes a form designer panel for creating a new simulation order template (e.g., as shown in FIG. 12). User selection of a template from the template library may also launch the second view of the template management GUI, with the sections and questions (as well as specified selections of options/answers for one or more of the questions) of the template displayed. In some examples, when a new template is created and the second view is launched, the form designer panel may include a default list of sections and/or questions for the new template.

Thus, if it is determined at 1504 that a user has requested to create or edit a simulation order template, method 1500 proceeds to 1506 to display the second view of the template management GUI including the form designer panel. At 1508, a question may be added to the simulation order template in response to user selection of a question from a question bank, such as question bank 1220. The question may be added to the bottom of the template and moved to a desired location on the template in response to user input (e.g., a drag and drop operation).

At 1510, a question on the template may be edited in response to user input to an options bar, such as options bar 1210. For example, a user may select a question on the template via the form designer panel, which may cause the options bar to be displayed with the current name of the question and the current set options (e.g., answers) for the question. Via the options bar, various parameters of the question may be edited. As indicated at 1512, editing the question may include editing the field name of the question (e.g., to therefore edit the name of the question). Additionally or alternatively, as indicated at 1514, editing the question may include editing the options for the question (e.g., possible answers/selections), such as editing an option name, adding a new option, deleting an option, and/or setting an option as a default choice. Other parameters for the question may be set via the options bar, including an arrangement of the options (e.g., tiled, list) whether or not the question is a mandatory question and whether the question should be hidden or visible. Further, as indicated at 1516, editing a question on the template may include creating a compounded question in response to user input to one or more hidden rules menus. For example, the options bar may include a link that when selected causes display of a hidden rules panel that includes a first menu (referred to as a choose menu) and a second menu (referred to as a show menu). The first menu may include the currently-set options for the selected question, and a user can select (e.g., choose) one of the options as a trigger for a subsequent question. The second menu may include the currently set-questions and the user can select one of the questions to be shown when the trigger option is chosen.

At 1518, a new question may be added to the question bank in response to user input to a tool bar, such as tool bar 1230. The tool bar may include user interface elements (e.g., buttons) that can be selected to add a new question, with each button specific to a type of question (e.g., single select, multiple select, text, number). Further, a new section may also be added to the template via the tool bar. Once a new question is added to the template, the new question may be edited via the options bar as explained above.

At 1520, method 1500 includes previewing and/or saving the template when requested. While the user is creating/editing the template in the manner described above, at any point the user may choose to preview the template by selecting a preview button (e.g., the preview element 1244). Similarly, at any point the user may choose to save the template by selecting a save button (e.g., the save form element 1242). Selection of the save button may cause the template to be saved in a non-relational database locally (e.g., on a device located at or otherwise exclusively accessible via the medical facility of the user). In some examples, a user may wish to delete a currently-saved template, and thus method 1500 may include deleting a template when requested. For example, via the first view of the template management GUI, a user may select a delete option (e.g., from a kabob menu) for a listed template in order to delete the template from the library (e.g., delete the template from the local non-relational database). It is to be appreciated that the aspects of creating and/or editing a template described above may be performed in any order and not all aspects may be performed each time a template is created and/or edited.

Returning to 1504, if the user has not requested to create or edit a simulation order template, method 1500 proceeds to 1524 to create or edit a plan intent template when requested. For example, responsive to user selection of the plan intent templates from the menu of the first view of the template management GUI, a template library of saved plan intent templates may be displayed and a “Create New Template” button may also be displayed. User selection of the “Create New Template” button may launch the second view of the template management GUI that includes the form designer panel for creating a new plan intent template. User selection of a template from the template library may also launch the second view of the template management GUI, with the sections and questions (as well as specified selections of options for one or more of the questions) of the template displayed. In some examples, when a new template is created and the second view is launched, the form designer panel may include a default list of sections and/or questions for the new template.

Creating or editing the plan intent template may include setting or adjusting parameters for a radiation therapy target and/or setting or adjusting parameters for guidelines, as indicated at 1526. For example, the plan intent template may include a specified radiation therapy target and the template shown in the form designer panel may include pre-specified menus and fields that the user may enter input to in order to set or adjust the parameters for the target, such as the radiation dose, beam type, expansion, coverage dose guidelines, etc. Further, creating or editing the plan intent template may include adding or removing a target, phase, and/or guide, as indicated at 1528. In at least some examples, editing of the plan intent templates may be limited to adjusting parameters and adding or removing targets, phases, and/or guidelines as explained above, and may not include options for adding other fields, sections, or questions to the template. Method 1500 may likewise include previewing and/or saving the plan intent template when requested at 1520 and deleting a plan intent template at 1522, which may be performed similarly as explained above.

FIG. 16 is a block diagram 1600 schematically showing an image workflow for RT planning, including a manual workflow 1602 and an automated workflow 1612. Each workflow includes steps/activities for performing a simulation scan, selecting images (e.g., CT images) from the simulation scan, and approving the selected images. Referring first to the manual workflow 1602, upon patient intake and scheduling of the simulation scan, the simulation scan may be performed so that CT images of the patient are acquired with a CT scanner (e.g., CT scanner 210), as shown at Acquire CT block 1604. The images acquired may be saved in an image archive (e.g., a PACS 1606). As explained previously, a user may later view the acquired images to select images for approval and eventual downstream tasks (e.g., contouring, segmentation) to facilitate RT delivery to desired targets. In the manual workflow 1602, the process of viewing the images obtained during the simulation scan and selecting the images, shown at Select CT block 1608, may be manual. For example, a user may query the PACS 1606 to identify exams/sets of images stored in the PACS for the patient, retrieve/view the images for the simulation scan, select the desired images, and initiate a manual transfer of the selected images for the subsequent task (e.g., storing the selected images in a data store 1610 and presenting the images for approval as shown by the Approve CT block 1611). The manual transfer may include the user specifying the device (e.g., data store 1610) to which the images are to be sent.

The automated workflow 1612 may be performed by the RT management system disclosed herein, as explained above. The automated workflow 1612 includes performance of the simulation scan so that CT images of the patient are acquired with a CT scanner (e.g., CT scanner 210), as shown at Acquire CT block 1614. The CT scanner may receive a modality worklist (MWL) that identifies the patient being scanned during the simulation scan and the order information (e.g., study instance UID and accession number). Prior to, during, and/or upon completion of the simulation scan, the CT scanner may be configured to generate and send one or more Modality Performed Procedure Step (MPPS) messages that describe the actual imaging procedure that was carried out during the simulation scan. The MPPS messages may identify the patient being imaged and identify the study/exam (e.g., by copying over the information from the MWL) as well as identify the actual procedure performed, radiation dose, and other information. The MPPS messages may also include a sequence of unique identifiers (e.g., instance IDs) that identify all DICOM objects generated during the acquisition, such as images. The MPPS messages may be sent to the RT management system so that an automatic query of the PACS may be performed by the RT management system to retrieve the images of the simulation scan, which may be stored in a data store 1616. When a user chooses to view the images from the simulation scan (e.g., during a “SelectCT” activity as explained above and as shown in the Select CT block 1618), the images from the simulation scan may be automatically presented to the user, without demanding that the user directly query the PACS. For example, when the user performs the Select CT activity, the simulation scan study/exam may be presented in a list of the input data panel of the activity detail view panel, and the images acquired during the simulation scan may be retrieved from the data store 1616 and viewed in response to user selection of the corresponding view button. Once the user has selected the images to be transferred for the next task, the selected images may be transferred automatically by the RT management system (e.g., transferred to a data store 1620 and presented for approval via the Approve CT block 1611).

The automated workflow may have advantages over the manual workflow. For example, automatically selecting the pertinent exam (and corresponding images) rather than relying on the user to query the PACS may avoid erroneous exam selection and hence avoid the user having to query the PACS multiple times, which may lower network traffic and improve the performance of the device communicating with the PACS to obtain the images. Due to the DICOM protocol, querying the PACS and moving DICOM objects (e.g., images) may be data-intensive processes and thus delays may be associated with the manual workflow as the images are not identified or moved until the user initiates the manual query. In contrast, via the automated workflow, the pertinent images may be identified automatically (e.g., via the MPPS messages) and stored in a centralized data store (e.g., data store 1616) to facilitate faster retrieval once the user requests to view the images. Further, some workflows may dictate that more than one activity be performed on a given image. For example, a user may decide that contouring should be performed on the same image two times, due to the image including multiple targets, for example. With the image pre-stored in the centralized data store, the user can perform the two activities on the image without having to perform two searches (e.g., a first search to find and retrieve the image for performing the first activity and a second search to re-find and retrieve the image for performing the second activity), lowering the processing demands placed on the devices. Further, the automated workflow may automatically identify and transfer selected images for the subsequent task, which may increase the speed at which the images are available for performing the next task and reduce the likelihood the images will be sent to the wrong device. As a non-limiting example, the contouring (or segmentation) module may be installed on three different computing devices, with each instance of the contouring (or segmentation) module configured for different contouring (or segmentation) functions (e.g., different anatomical regions or different contouring/segmenting tasks). The RT management system may perform automatic data transfers based on workflow models that take into account which devices are best suited to perform which tasks. As such, the automatic transfer may reduce instances of the images being transferred to the wrong device, which may lower network traffic and improve device performance.

Thus, a computing device comprising a display screen is disclosed herein, the computing device being configured to display on the display screen a menu listing one or more applications, and additionally being configured to display on the display screen a course directive graphical user interface (GUI) that can be reached directly from the menu. The course directive GUI may display, for a selected patient, a limited list of patient information, the limited list of patient information obtained from one or more of an EMR database and an OIS. The course directive GUI may further display a limited list of radiation therapy forms, each radiation therapy form in the limited list of radiation therapy forms being selectable to launch a display panel and enable at least the selected form to be seen within the display panel, and wherein the course directive GUI is displayed while the one or more applications are in an un-launched state.

Thus, when the course directive GUI is displayed, the one or more applications may exist in an un-launched state (e.g., not displayed and not currently access by the display device). Further, the data from the EMR database and/or OIS may be used to populate the patient information portion of the course directive GUI and used to populate the radiation therapy forms (e.g., the simulation order form and plan intent form) displayed in the additional display panels without the user having to access the EMRs and/or OIS records themselves (e.g., in a separate window). In doing so, the course directive GUI as disclosed herein may present a limited set of information (e.g., patient information and radiation therapy forms) to the users/care providers in order to increase efficiency of the users' interaction with the available data, as users are not forced to sift through multiple separate EMRs, OIS records/interfaces, data feeds, data files, etc., to identify and then aggregate the needed information.

Further, the computing device may be configured to display on the display screen a multi-patient GUI, wherein the multi-patient GUI displays, for each patient of a plurality of patients, a limited list of patient information, the limited list of patient information obtained from one or more of an electronic medical record (EMR) database and an oncology information system (OIS), the multi-patient GUI further displaying a workflow for each patient visually representing a list of activities to be performed in order to delivery radiation therapy to each patient, each activity of the workflow being selectable to launch an activity detail view display panel and enable the activity to be performed via the display panel, and wherein at least one activity of the workflow includes user selection of medical images stored in an image archive, the medical images displayed via an image viewer launched automatically in response to user input to the detail view display panel. For example, the radiation therapy management system disclosed herein may receive an indication that a user is performing an activity where the user will select medical images to be transferred from the image archive (e.g., the PACS) to a segmentation module, for example. Based on the identifying information of the patient, the radiation therapy management system may automatically identify the medical images pertinent to the delivery of radiation therapy, such as the CT images acquired during the simulation scan for the patient, and list the pertinent medical images for selection on the activity detail view display panel (e.g., by exam). In response to user selection of the pertinent medical images (e.g., selection of an exam) from the list, the medical images may be displayed via the image viewer. By providing the workflow via the multi-patient GUI with each activity being selectable to cause display of the activity detail view display panel, the user may be prompted to perform the next activity in the workflow and may be able to actually perform the activity without having to navigate to a separate interface, such as an interface for viewing images via the image archive, and perform a search to find the pertinent medical images.

Additionally, the computing device may be configured to display on the display screen a menu listing one or more applications, and additionally being configured to display on the display screen a template management graphical user interface (GUI) that can be reached directly from the menu, wherein the template management GUI displays a list of stored radiation therapy form templates, the template management GUI further displaying a limited list of radiation therapy forms, each radiation therapy form template in the list of radiation therapy form templates being selectable to launch a form designer display panel and enable at least the selected radiation therapy form template to be seen and edited within the display panel, and wherein the template management GUI is displayed while the one or more applications are in an un-launched state.

The systems, methods, and graphical user interfaces provided herein may provide a technical effect of improved accuracy and timeliness of radiation therapy delivery, which may be particularly useful during high-demand situations where time is limited. The time it would take to individually collect data from multiple EMRs and OIS records and visualize the data using standard methods could render the data useless by the time the data was visualized, because patient conditions may change during the data collection and visualization. By establishing a system that automatically retrieves data from all EMRs and/or OIS records in real time or near real time, aggregates that information regardless of data format, and continually updates the course directive GUI and multi-patient GUI as data is received, the approach of the disclosure allows care providers to make informed decisions about radiation therapy, such as where to deliver the radiation therapy and at what dose, thereby improving patient care.

In contrast, in prior systems when one or more care providers would plan a simulation scan and radiation therapy delivery for a patient, errors and/or delays could be made due to delays in individual care providers obtaining all necessary parameters for evaluating a patient's diagnosis and appropriately planning the simulation scan and radiation therapy delivery. For example, a patient's condition may have changed to the point where the patient requires different radiation therapy parameters, but limited care provider resources may result in the patient's treatment being delayed, or inaccurate information may be entered owing to the requirement that redundant patient information be obtained/manually entered. The described course directive and multi-patient GUIs address this problem and provide a further technical effect by bringing together information from multiple, disparate systems (e.g., EMR database and OIS, PACS), auto-populating radiation therapy forms when relevant patient information is available, displaying a human figure with relevant medical information of the patient (e.g., pacemaker location, treatment ROI), automatically launching external interfaces (e.g., DICOM viewer; segmentation interface) and/or allowing external patient information (e.g., stored in a PACS) to be viewed directly via the GUI, and pushing out information obtained via the radiation therapy forms to multiple other, disparate systems (e.g., CT scanner and radiation therapy device). In this way, the course directive and multi-patient GUIs provide an improvement to the capability of the healthcare system as a whole. The disclosure provides a specific way of improving the capability of the healthcare system, by providing a course directive GUI that displays radiation therapy forms (and summaries thereof) for multiple different modalities (e.g., CT scanner and LINAC system). The disclosure further provides a specific improvement to the way computers operate by aggregating EMR data and OIS data for a patient in one location, which may obviate the need for users to have to navigate through multiple different EMRs/OIS data files, manually enter information into multiple radiation therapy planning forms, and so forth, thereby increasing the efficiency of the operation of the computer for the user.

The course directive GUI described herein provides a technical effect of a specific manner of displaying a limited set of information to a user (patient information and radiation therapy planning forms), rather than using conventional user interface methods to display a generic index on a computer, requiring the user to step through many layers of menu options to access the desired data, or burying the desired data within all hospital data. Thus, the user experience with the computer may be improved and made more efficient.

Furthermore, by displaying a limited set of information via the GUIs as described herein, a technical effect of an operation of the computing device(s) that collect and render the data for display may be improved by reducing the processing demands of the computing device(s), thereby increasing the efficiency of the computing device(s). For example, only certain patient information relevant to the radiation therapy may be displayed and previously-entered information may be used to auto-populate fields of the radiation therapy forms, which results in a limited amount of the data that is received being processed, which may improve the efficiency of the computing device(s). Further, in some examples, the data is processed in real time and updates to the GUIs (e.g., the multi-patient GUI) are made continuously as data is received, and therefore undue processing lags that occur if the updates were made at predefined time points may be reduced, which may improve the efficiency of the computing device(s).

Thus, via the disclosed radiation therapy management system and GUIs, a technical effect of patient information and radiation therapy forms (and summaries thereof) being displayed in a manner that is easy to visually parse and act on in a reduced amount of time is provided. The patient information may be stored in different data repositories that would otherwise be accessed via individual interfaces, and by using the radiation therapy management system to aggregate the patient information, an amount of time necessary to review relevant patient information for diagnosis and treatment decisions may be reduced. The radiation therapy management system disclosed herein may also aggregate patient data to a single place, into a single application, which may help to reduce wasted time spent searching for known but scattered data, and unknown and missing data. This may reduce cognitive overloads and aid clinical thinking; because the patient data is reconstructed into a clinically helpful structure.

The disclosure also provides support for a computing device comprising a display screen, the computing device being configured to display on the display screen a course directive graphical user interface (GUI), wherein the course directive GUI displays, for a selected patient, a limited list of patient information, the limited list of patient information obtained from one or more of an electronic medical record (EMR) database and an oncology information system (OIS), the course directive GUI further displaying a limited list of radiation therapy forms, each radiation therapy form in the limited list of radiation therapy forms being selectable to launch a display panel and enable at least the selected radiation therapy form to be seen within the display panel. In a first example of the system, the limited list of radiation therapy forms includes a simulation order form and a plan intent form, wherein the simulation order form includes a first plurality of fillable fields for planning a simulation scan of the selected patient and the plan intent form includes a second plurality of fillable fields for planning one or more radiation therapy sessions of the selected patient. In a second example of the system, optionally including the first example, the course directive GUI is configured to display a first summary of information entered into the simulation order form and a second summary of information entered into the plan intent form. In a third example of the system, optionally including one or both of the first and second examples, the course directive GUI further includes a human figure, wherein the human figure includes highlighting positioned based on the limited list of patient information and/or based on information obtained with the limited list of radiation therapy forms. In a fourth example of the system, optionally including one or more or each of the first through third examples, the limited list of radiation therapy forms includes a simulation order form and a plan intent form that are each displayed via a respective display panel viewable simultaneously on the course directive GUI, wherein the human figure is displayed intermediate the respective display panels. In a fifth example of the system, optionally including one or more or each of the first through fourth examples, the computing device is further configured to display on the display screen a multi-patient GUI, wherein the multi-patient GUI displays, for each patient of a plurality of patients including the selected patient, a workflow visually representing a list of activities to be performed in order to delivery radiation therapy to each patient, each activity of the workflow being selectable to launch an activity detail view display panel and enable the activity to be performed via the display panel, and wherein at least one activity of a selected workflow includes user selection of medical images stored in an image archive, the medical images displayed via an image viewer launched automatically in response to user input to the activity detail view display panel. In a sixth example of the system, optionally including one or more or each of the first through fifth examples, the selected workflow is for the selected patient, wherein the medical images are obtained during the simulation scan for the selected patient. In a seventh example of the system, optionally including one or more or each of the first through sixth examples, the course directive GUI is launched in response to user input to the multi-patient GUI.

The disclosure also provides support for a method for a radiation therapy management system, comprising: displaying a course directive graphical user interface (GUI) that displays, for a selected patient, a human figure, patient information in an intake information panel, and a limited list of radiation therapy forms, wherein at least some of the patient information displayed in the course directive GUI is obtained from an electronic medical record (EMR) database and/or an oncology information system (OIS), in response to a selection of a simulation order form of the limited list of radiation therapy forms, displaying the simulation order form in a first display panel, receiving user input filling one or more fields of a plurality of fields of the simulation order form, wherein at least some of the plurality of fields of the simulation order form are auto-populated with patient information from the intake information panel, displaying a multi-patient GUI that displays a workflow for each patient of a plurality of patients including the selected patient, updating a workflow progress bar for the selected patient of the multi-patient GUI upon receiving the user input filling the one or more fields of the simulation order form, in response to a selection of a plan intent form of the limited list of radiation therapy forms, displaying the plan intent form in a second display panel, and receiving user input filling one or more fields of a plurality of fields of the plan intent form, wherein at least some of the plurality of fields of the plan intent form are auto-populated with patient information from the intake information panel and/or with information from the simulation order form. In a first example of the method, the method further comprises: upon receiving the user input filling the one or more fields of a plurality of fields of the simulation order form, sending the filled simulation order form to a computed tomography imaging system. In a second example of the method, optionally including the first example, the computed tomography imaging system displays an imaging interface and the filled simulation order form is viewable on the imaging interface. In a third example of the method, optionally including one or both of the first and second examples, the workflow progress bar is displayed as part of a selected workflow for the selected patient on the multi-patient GUI, and further comprising displaying, on the multi-patient GUI, an activity detail view display panel in response to user input to an activity of the selected workflow, the activity detail view display panel displaying a list of medical images obtained via the computed tomography imaging system and stored in an image archive, the medical images configured to be displayed via an image viewer launched automatically in response to user input to the activity detail view display panel. In a fourth example of the method, optionally including one or more or each of the first through third examples, the method further comprises: upon receiving the user input filling the one or more fields of a plurality of fields of the plan intent form, sending the filled plan intent form to a radiation therapy device. In a fifth example of the method, optionally including one or more or each of the first through fourth examples, the human figure is displayed intermediate the first display panel and the second display panel, and further comprising highlighting a treatment region of interest on the human figure in response to identification of the treatment region of interest via the patient information in the intake information panel, the simulation order form, and/or the plan intent form.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A computing device comprising a display screen, the computing device being configured to display on the display screen a course directive graphical user interface (GUI), wherein the course directive GUI displays, for a selected patient, a limited list of patient information, the limited list of patient information obtained from one or more of an electronic medical record (EMR) database and an oncology information system (OIS), the course directive GUI further displaying a limited list of radiation therapy forms, each radiation therapy form in the limited list of radiation therapy forms being selectable to launch a display panel and enable at least the selected radiation therapy form to be seen within the display panel.

2. The computing device of claim 1, wherein the limited list of radiation therapy forms includes a simulation order form and a plan intent form, wherein the simulation order form includes a first plurality of fillable fields for planning a simulation scan of the selected patient and the plan intent form includes a second plurality of fillable fields for planning one or more radiation therapy sessions of the selected patient.

3. The computing device of claim 2, wherein the course directive GUI is configured to display a first summary of information entered into the simulation order form and a second summary of information entered into the plan intent form.

4. The computing device of claim 1, wherein the course directive GUI further includes a human figure, wherein the human figure includes highlighting positioned based on the limited list of patient information and/or based on information obtained with the limited list of radiation therapy forms.

5. The computing device of claim 4, wherein the limited list of radiation therapy forms includes a simulation order form and a plan intent form that are each displayed via a respective display panel viewable simultaneously on the course directive GUI, wherein the human figure is displayed intermediate the respective display panels.

6. The computing device of claim 2, wherein the computing device is further configured to display on the display screen a multi-patient GUI, wherein the multi-patient GUI displays, for each patient of a plurality of patients including the selected patient, a workflow visually representing a list of activities to be performed in order to delivery radiation therapy to each patient, each activity of the workflow being selectable to launch an activity detail view display panel and enable the activity to be performed via the display panel, and wherein at least one activity of a selected workflow includes user selection of medical images stored in an image archive, the medical images displayed via an image viewer launched automatically in response to user input to the activity detail view display panel.

7. The computing device of claim 6, wherein the selected workflow is for the selected patient, wherein the medical images are obtained during the simulation scan for the selected patient.

8. The computing device of claim 6, wherein the course directive GUI is launched in response to user input to the multi-patient GUI.

9. A method for a radiation therapy management system, comprising:
displaying a course directive graphical user interface (GUI) that displays, for a selected patient, a human figure, patient information in an intake information panel, and a limited list of radiation therapy forms, wherein at least some of the patient information displayed in the course directive GUI is obtained from an electronic medical record (EMR) database and/or an oncology information system (OIS);
in response to a selection of a simulation order form of the limited list of radiation therapy forms, displaying the simulation order form in a first display panel;
receiving user input filling one or more fields of a plurality of fields of the simulation order form, wherein at least some of the plurality of fields of the simulation order form are auto populated with patient information from the intake information panel;
displaying a multi-patient GUI that displays a workflow for each patient of a plurality of patients including the selected patient;
updating a workflow progress bar for the selected patient of the multi-patient GUI upon receiving the user input filling the one or more fields of the simulation order form;
in response to a selection of a plan intent form of the limited list of radiation therapy forms, displaying the plan intent form in a second display panel; and
receiving user input filling one or more fields of a plurality of fields of the plan intent form, wherein at least some of the plurality of fields of the plan intent form are auto-populated with patient information from the intake information panel and/or with information from the simulation order form.

10. The method of claim 9, further comprising, upon receiving the user input filling the one or more fields of a plurality of fields of the simulation order form, sending the filled simulation order form to a computed tomography imaging system.

11. The method of claim 10, wherein the computed tomography imaging system displays an imaging interface and the filled simulation order form is viewable on the imaging interface.

12. The method of claim 10, wherein the workflow progress bar is displayed as part of a selected workflow for the selected patient on the multi-patient GUI, and further comprising displaying, on the multi-patient GUI, an activity detail view display panel in response to user input to an activity of the selected workflow, the activity detail view display panel displaying a list of medical images obtained via the computed tomography imaging system and stored in an image archive, the medical images configured to be displayed via an image viewer launched automatically in response to user input to the activity detail view display panel.

13. The method of claim 9, further comprising, upon receiving the user input filling the one or more fields of a plurality of fields of the plan intent form, sending the filled plan intent form to a radiation therapy device.

14. The method of claim 9, wherein the human figure is displayed intermediate the first display panel and the second display panel, and further comprising highlighting a treatment region of interest on the human figure in response to identification of the treatment region of interest via the patient information in the intake information panel, the simulation order form, and/or the plan intent form.

15. A non-transitory computer-executable medium comprising a plurality of instructions that, in response to execution by a processor, cause the processor to:
display a course directive graphical user interface (GUI) that displays, for a selected patient, a human figure, patient information in an intake information panel, and a limited list of radiation therapy forms, wherein at least some of the patient information displayed in the course directive GUI is obtained from an electronic medical record (EMR) database and/or an oncology information system (OIS);
in response to a selection of a simulation order form of the limited list of radiation therapy forms, display the simulation order form in a first display panel;
receive user input filling one or more fields of a plurality of fields of the simulation order form, wherein at least some of the plurality of fields of the simulation order form are auto populated with patient information from the intake information panel;
display a multi-patient GUI that displays a workflow for each patient of a plurality of patients including the selected patient;
update a workflow progress bar for the selected patient of the multi-patient GUI upon receiving the user input filling the one or more fields of the simulation order form;
in response to a selection of a plan intent form of the limited list of radiation therapy forms, display the plan intent form in a second display panel; and
receive user input filling one or more fields of a plurality of fields of the plan intent form, wherein at least some of the plurality of fields of the plan intent form are auto-populated with patient information from the intake information panel and/or with information from the simulation order form.

16. The non-transitory computer-executable medium of claim 15, wherein the plurality of instructions cause the processor to, upon receiving the user input filling the one or more fields of a plurality of fields of the simulation order form, send the filled simulation order form to a computed tomography imaging system.

17. The non-transitory computer-executable medium of claim 16, wherein the computed tomography imaging system displays an imaging interface and the filled simulation order form is viewable on the imaging interface.

18. The non-transitory computer-executable medium of claim 16, wherein the workflow progress bar is displayed as part of a selected workflow for the selected patient on the multi-patient GUI, and further comprising displaying, on the multi-patient GUI, an activity detail view display panel in response to user input to an activity of the selected workflow, the activity detail view display panel displaying a list of medical images obtained via the computed tomography imaging system and stored in an image archive, the medical images configured to be displayed via an image viewer launched automatically in response to user input to the activity detail view display panel.

19. The non-transitory computer-executable medium of claim 15, wherein the plurality of instructions cause the processor to, upon receiving the user input filling the one or more fields of a plurality of fields of the plan intent form, send the filled plan intent form to a radiation therapy device.

20. The non-transitory computer-executable medium of claim 15, wherein the human figure is displayed intermediate the first display panel and the second display panel, and further comprising highlighting a treatment region of interest on the human figure in response to identification of the treatment region of interest via the patient information in the intake information panel, the simulation order form, and/or the plan intent form.
